SEQUENCE LISTING (63 sequences)

SEQ ID NO: 1 - RNA, 8 nt, Artificial Sequence
  synthetic nucleotide
gucauaca

SEQ ID NO: 2 - RNA, 8 nt, Artificial Sequence
  synthetic nucleotide
ucauacac

SEQ ID NO: 3 - RNA, 9 nt, Artificial Sequence
  synthetic nucleotide
ucauacacg

SEQ ID NO: 4 - RNA, 10 nt, Artificial Sequence
  synthetic nucleotide
ucauacacgg

SEQ ID NO: 5 - RNA, 11 nt, Artificial Sequence
  synthetic nucleotide
ucauacacggc

SEQ ID NO: 6 - RNA, 12 nt, Artificial Sequence
  synthetic nucleotide
ucauacacggcu

SEQ ID NO: 7 - RNA, 13 nt, Artificial Sequence
  synthetic nucleotide
ucauacacggcuc

SEQ ID NO: 8 - RNA, 14 nt, Artificial Sequence
  synthetic nucleotide
ucauacacggcucu

SEQ ID NO: 9 - RNA, 15 nt, Artificial Sequence
  synthetic nucleotide
ucauacacggcucuc

SEQ ID NO: 10 - RNA, 16 nt, Artificial Sequence
  synthetic nucleotide
ucauacacggcucucc

SEQ ID NO: 11 - RNA, 17 nt, Artificial Sequence
  synthetic nucleotide
ucauacacggcucuccu

SEQ ID NO: 12 - RNA, 18 nt, Artificial Sequence
  synthetic nucleotide
ucauacacggcucuccuc

SEQ ID NO: 13 - RNA, 19 nt, Artificial Sequence
  synthetic nucleotide
ucauacacggcucuccucu

SEQ ID NO: 14 - RNA, 19 nt, Artificial Sequence
  synthetic nucleotide
ucauacacggcucuccucu

SEQ ID NO: 15 - RNA, 20 nt, Artificial Sequence
  synthetic nucleotide
ucauacacggcucuccucuc

SEQ ID NO: 16 - RNA, 21 nt, Artificial Sequence
  synthetic nucleotide
ucauacacggcucuccucucu

SEQ ID NO: 17 - RNA, 9 nt, Artificial Sequence
  synthetic nucleotide
gucauacac

SEQ ID NO: 18 - RNA, 10 nt, Artificial Sequence
  synthetic nucleotide
gucauacacg

SEQ ID NO: 19 - RNA, 11 nt, Artificial Sequence
  synthetic nucleotide
gucauacacgg

SEQ ID NO: 20 - RNA, 12 nt, Artificial Sequence
  synthetic nucleotide
gucauacacggc

SEQ ID NO: 21 - RNA, 13 nt, Artificial Sequence
  synthetic nucleotide
gucauacacggcu

SEQ ID NO: 22 - RNA, 14 nt, Artificial Sequence
  synthetic nucleotide
gucauacacggcuc

SEQ ID NO: 23 - RNA, 15 nt, Artificial Sequence
  synthetic nucleotide
gucauacacggcucu

SEQ ID NO: 24 - RNA, 16 nt, Artificial Sequence
  synthetic nucleotide
gucauacacggcucuc

SEQ ID NO: 25 - RNA, 17 nt, Artificial Sequence
  synthetic nucleotide
gucauacacggcucucc

SEQ ID NO: 26 - RNA, 18 nt, Artificial Sequence
  synthetic nucleotide
gucauacacggcucuccu

SEQ ID NO: 27 - RNA, 19 nt, Artificial Sequence
  synthetic nucleotide
gucauacacggcucuccuc

SEQ ID NO: 28 - RNA, 20 nt, Artificial Sequence
  synthetic nucleotide
gucauacacggcucuccucu

SEQ ID NO: 29 - skipped

SEQ ID NO: 30 - RNA, 21 nt, Artificial Sequence
  synthetic nucleotide
gucauacacggcucuccucuc

SEQ ID NO: 31 - RNA, 22 nt, Artificial Sequence
  synthetic nucleotide
gucauacacggcucuccucucu

SEQ ID NO: 32 - RNA, 7 nt, Artificial Sequence
  synthetic nucleotide
ucauaca

SEQ ID NO: 33 - RNA, 73 nt, Artificial Sequence
  synthetic nucleotide
acuuggagagaggcuggccgugaugaauucgauucaucaaagcgagucauacacggcucuccucucuuuuuuu

SEQ ID NO: 34 - RNA, 22 nt, Artificial Sequence
  synthetic nucleotide
agucauacacggcucuccucuc

SEQ ID NO: 35 - RNA, 22 nt, Artificial Sequence
  synthetic nucleotide
gucauacacggcucuccucucu

SEQ ID NO: 36 - skipped

SEQ ID NO: 37 - DNA, 22 bp, Artificial Sequence
  synthetic nucleotide
gtcatacacggctctcctctct

SEQ ID NO: 38 - DNA, 17 bp, Artificial Sequence
  synthetic nucleotide
gtcatacacggctctcc

SEQ ID NO: 39 - DNA, 18 bp, Artificial Sequence
  synthetic nucleotide
gtcatacacggctctcct

SEQ ID NO: 40 - DNA, 19 bp, Artificial Sequence
  synthetic nucleotide
gtcatacacggctctcctc

SEQ ID NO: 41 - DNA, 20 bp, Artificial Sequence
  synthetic nucleotide
gtcatacacggctctcctct

SEQ ID NO: 42 - DNA, 21 bp, Artificial Sequence
  synthetic nucleotide
gtcatacacggctctcctctc

SEQ ID NO: 43 - DNA, 1813 bp, Artificial Sequence
  synthetic nucleotide
tgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaacttaaaaaaaaaaaaaaaaa

SEQ ID NO: 44 - DNA, 500 bp, Artificial Sequence
  synthetic nucleotide
ccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtg

SEQ ID NO: 45 - DNA, 7 bp, Artificial Sequence
  synthetic nucleotide
tgtatga

SEQ ID NO: 46 - DNA, 7 bp, Artificial Sequence
  synthetic nucleotide
agatagt

SEQ ID NO: 47 - DNA, 73 bp, Artificial Sequence
  synthetic nucleotide
acttggagagaggctggccgtgatgaattcgattcatcaaagcgagtcatacacggctctcctctcttttagt

SEQ ID NO: 48 - DNA, 115 bp, Artificial Sequence
  synthetic nucleotide
caagtgtccacttatggtacttgaagagaggctggccgtgatgaattcgattcatcaaagcgagtcatacacggctctcctctcttttagtgtcaaattctgcctcggagggctt

SEQ ID NO: 49 - DNA, 111 bp, Artificial Sequence
  synthetic nucleotide
caagtgtccacttatggtacttgaagagaggctggccgtgatgaattcgattcatcaaagcgagtcatacacggctctcctctcttttagtgtcaaattctgcctcggagg

SEQ ID NO: 50 - DNA, 109 bp, Artificial Sequence
  synthetic nucleotide
ttcatgtgtggtacttggagagaggctggccgtgatgaattcgattcatcaaagcgagtcatacacggctctcctctcttttagtgtcaaattctgcctcggagggctt

SEQ ID NO: 51 - DNA, 79 bp, Artificial Sequence
  synthetic nucleotide
gatacttggagagaggctggccgtgatgaattcgattcatctaaacgagtcatacacggctctcctctcttctagtgtc

SEQ ID NO: 52 - DNA, 79 bp, Artificial Sequence
  synthetic nucleotide
gtacttggagagaggctggccgtgatgaattcgattcatcaaagcgagtcatacacggctctcctctcttttagtatca

SEQ ID NO: 53 - DNA, 73 bp, Artificial Sequence
  synthetic nucleotide
acttggagagaggctggccgtgatgaattcgattcatctaaacgagtcatacacggctctcctctcttctagt

SEQ ID NO: 54 - DNA, 73 bp, Artificial Sequence
  synthetic nucleotide
acttgaagagaggctggccgtgatgaattcgattcatcaaagcgagtcatacacggctctcctctcttttagt

SEQ ID NO: 55 - DNA, 73 bp, Artificial Sequence; identical to SEQ ID NO: 47
  synthetic nucleotide

SEQ ID NO: 56 - DNA, 73 bp, Artificial Sequence; identical to SEQ ID NO: 47
  synthetic nucleotide

SEQ ID NO: 57 - DNA, 73 bp, Artificial Sequence; identical to SEQ ID NO: 54
  synthetic nucleotide

SEQ ID NO: 58 - DNA, 72 bp, Artificial Sequence
  synthetic nucleotide
cttggagagaggctggccgtgatgaattcgattcatcaaagcgagtcatacacggctctcctctcttttagt

SEQ ID NO: 59 - DNA, 59 bp, Artificial Sequence
  synthetic nucleotide
agaggctggccgtgatgaattcgattcatcaaagcgagtcatacacggctctcctctct

SEQ ID NO: 60 - DNA, 59 bp, Artificial Sequence
  synthetic nucleotide
agaggctggccatggtgaattcgattcatcaaagcgagtcatacacggctctcctctct

SEQ ID NO: 61 - DNA, 59 bp, Artificial Sequence; identical to SEQ ID NO: 60
  synthetic nucleotide

SEQ ID NO: 62 - DNA, 59 bp, Artificial Sequence; identical to SEQ ID NO: 59
  synthetic nucleotide

SEQ ID NO: 63 - DNA, 59 bp, Artificial Sequence; identical to SEQ ID NO: 59
  synthetic nucleotide

(19) United States
(12) Patent Application Publication
(10) Pub. No.: US 2023/0256119 A1
(43) Pub. Date: Aug. 17, 2023

(54) COMPOSITIONS AND METHODS FOR PREPARING AN ALZHEIMER'S DISEASE ANIMAL MODEL USING MICRORNA

(71) Applicant: Biorchestra Co., Ltd., Daejeon (KR)

(72) Inventors: Jin-Hyeob RYU, Daejeon (KR); Hyo Jin LEE, Daejeon (KR); Hyun Su MIN, Daejeon (KR); Sang Moo LEE, Daejeon (KR)

(73) Assignee: Biorchestra Co., Ltd., Daejeon (KR)

(21) Appl. No.: 17/620,549

(22) PCT Filed: Jun. 17, 2020
(86) PCT No.: PCT/IB2020/055670

§ 371 Date: Dec. 17, 2021

Related U.S. Application Data
(60) Provisional application No. 62/862,586, filed on Jun. 17, 2019.

(51) Int. Cl. A61K 49/00; C12N 15/113; A61P 25/28

(52) U.S. Cl. CPC A61K 49/0008; C12N 15/113; A61P 25/28; C12N 2310/141

(57) ABSTRACT

The present invention relates to a composition for preparing an Alzheimer's disease animal model using microRNA, a non-human Alzheimer's disease animal model, and a method for screening compounds capable of treating Alzheimer's disease using the same.

### CROSS-REFERENCE TO RELATED APPLICATIONS

This application claims the priority benefit of U.S. Provisional Application No. 62/862,586, filed Jun. 17, 2019 and is hereby incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4366_007PC01_Seqlisting_ST25; Size: 16,187 bytes, and Date of Creation: Jun. 17, 2020) filed with the application is herein incorporated by reference in its entirety.

### BACKGROUND OF THE DISCLOSURE

Alzheimer's disease (AD) is the most common form of dementia, where 75% of Alzheimer's patients have dementia. In most cases, Alzheimer's disease occurs in people above 65 years of age, though it can also occur before that age. In the United States, about 3% of the population aged 65 to 74, about 19% of the population aged 75 to 84, and about 50% of the population aged 85 or older suffer from the disease. According to a recent report in a rural area in Korea, about 21% of people aged 60 years or older in the rural area show dementia, and 63% of them are reported to be dementia due to Alzheimer's disease. In 2050, one out of every 85 people is expected to develop Alzheimer's disease.

The appearance of senile plaques due to the accumulation of amyloid beta (Aβ) is the most prominent feature of Alzheimer's disease and this disease can be confirmed by post-mortem examination (Khachaturian, Arch. Neurol. 42 (11): 1097-105)). The process of accumulation of amyloid beta in the neuronal signal transduction pathways is not clear, and when APP is abnormally cleaved to produce amyloid beta and accumulate in the neuritic space, plaque formation is induced. Many other factors involved in this cleavage reaction (such as inflammatory responses) also increase the phosphorylation of Tau protein and may result in the accumulation of neurofibrillary tangles (NFTs) and paired helical filaments (PHFs) eventually leading to damage of the nerve. All of these factors lead to nerve dysfunction and ultimately accelerate the progression of Alzheimer's disease to dementia.

However, there have been a long line of clinical trial failures in developing the therapeutics for Alzheimer's disease. From 1998 to 2017, there have been about 146 failed attempts at developing Alzheimer's drugs, and 2018 marked another half-dozen or so. Therefore, there is a significant need for effective tools, e.g., animal model, to identify drugs for treating Alzheimer's disease.

### SUMMARY OF THE DISCLOSURE

The present disclosure is directed to an animal model of Alzheimer's disease that shows the pathological features of Alzheimer's disease. This model shows critical features of Alzheimer's disease, including amyloid beta accumulation in normal mice treated with miR-485-3p analogue, neuropathy, neuroinflammation, behavioral impairment, and memory depression.

In some aspects, a present method comprises preparing a non-human animal model for Alzheimer's disease comprising administering to a non-human animal a compound that mimics miR-485 (miRNA compound). The miRNA compound of the present disclosure mimics the role of miR-485-3p, thereby exhibiting an effect of increasing expression or activation of miR-485-3p. In some aspects, the miRNA compound comprises a nucleotide sequence comprising 5′ UCAUACA 3′ (SEQ ID NO: 32) and wherein the miRNA compound comprises about 6 to about 30 nucleotides in length. In some aspects, the miRNA compound reduces transcription of an SIRT1 gene and/or expression of a SIRT1 protein. In some aspects, the miRNA compound comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, the miRNA compound comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence. In some aspects, the miRNA compound has a sequence selected from the group consisting of:

| (SEQ ID NO: 1) | |
|---|---|
| | GUCAUACA, |
| (SEQ ID NO: 2) | |
| | UCAUACAC, |
| (SEQ ID NO: 3) | |
| | UCAUACACG, |
| (SEQ ID NO: 4) | |
| | UCAUACACGG, |
| (SEQ ID NO: 5) | |
| | UCAUACACGGC, |
| (SEQ ID NO: 6) | |
| | UCAUACACGGCU, |
| (SEQ ID NO: 7) | |
| | UCAUACACGGCUC, |
| (SEQ ID NO: 8) | |
| | UCAUACACGGCUCU, |
| (SEQ ID NO: 9) | |
| | UCAUACACGGCUCUC, |
| (SEQ ID NO: 10) | |
| | UCAUACACGGCUCUCC, |
| (SEQ ID NO: 11) | |
| | UCAUACACGGCUCUCCU, |
| (SEQ ID NO: 12) | |
| | UCAUACACGGCUCUCCUC, |
| (SEQ ID NO: 13) | |
| | UCAUACACGGCUCUCCUCU, |
| (SEQ ID NO: 14) | |
| | UCAUACACGGCUCUCCUCU, |
| (SEQ ID NO: 15) | |
| | UCAUACACGGCUCUCCUCUC, |
| (SEQ ID NO: 16) | |
| | UCAUACACGGCUCUCCUCUCU, |
| (SEQ ID NO: 17) | |
| | GUCAUACAC, |
| (SEQ ID NO: 18) | |
| | GUCAUACACG, |
| (SEQ ID NO: 19) | |
| | GUCAUACACGG, |
| (SEQ ID NO: 20) | |
| | GUCAUACACGGC, |
| (SEQ ID NO: 21) | |
| | GUCAUACACGGCU, |
| (SEQ ID NO: 22) | |
| | GUCAUACACGGCUC, |
| (SEQ ID NO: 23) | |
| | GUCAUACACGGCUCU, |
| (SEQ ID NO: 24) | |
| | GUCAUACACGGCUCUC, |
| (SEQ ID NO: 25) | |
| | GUCAUACACGGCUCUCC, |
| (SEQ ID NO: 26) | |
| | GUCAUACACGGCUCUCCU, |
| (SEQ ID NO: 27) | |
| | GUCAUACACGGCUCUCCUC, |
| (SEQ ID NO: 28) | |
| | GUCAUACACGGCUCUCCUCU, |
| (SEQ ID NO: 30) | |
| | GUCAUACACGGCUCUCCUCUC, |
| | and |
| (SEQ ID NO: 31) | |
| | GUCAUACACGGCUCUCCUCUCU. |

In some aspects, the sequence of the miRNA compound is at least about 50%, at least about 55%, at least about 60%, at least about 65%, at least about 70%, at least about 75%, at least about 80%, at least about 85%, at least about 90%, or at least about 95% sequence identity to GUCAUACACGGCUCUCCUCUCU (SEQ ID NO: 31). In some aspects, the miRNA compound has a sequence that has at least 90% similarity to GUCAUACACGGCUCUCCUCUCU (SEQ ID NO: 31). In some aspects, the miRNA compound comprises the nucleotide sequence GUCAUACACGGCUCUCCUCUCU (SEQ ID NO: 31) with one substitution or two substitutions. In some aspects, the miRNA compound comprises the nucleotide sequence GUCAUACACGGCUCUCCUCUCU (SEQ ID NO: 31). In some aspects, the miRNA compound comprises at least one modified nucleotide. in some aspects, the at least one modified nucleotide is a locked nucleic acid (LNA), an unlocked nucleic acid (UNA), an arabino nucleic acid (ABA), a bridged nucleic acid (BNA), and/or a peptide nucleic acid (PNA). In some aspects, the miRNA compound comprises a backbone modification, in some aspects, the backbone modification is a phosphorodiamidate morpholino oligomer (PMO) and/or phosphorothioate (PS) modification.

The mothods of the present disclosure are useful for generating an animal model capable of displaying symptoms of Alzheimer's disease. Such models can then be used in the furtherance of therapeutics to treat Alzheimer's disease or other neurodegenerative disease. The present disclosure is related to a method of preparing a non-human animal model for Alzheimer's disease comprising administering to a non-human animal a miRNA compound that mimics miR-485-3p (“miRNA compound”). In some aspects, the present disclosure is related to a method of preparing a non-human animal model for Alzheimer's disease comprising administering to a non-human animal a miRNA compound that inhibit or reduce expression of a SIRT1 protein or a SIRT1 mRNA. In some aspects, the miRNA compound that inhibit or reduce expression of a SIRT1 protein or a SIRT1 mRNA is miR-485 or miR-485 mimic.

The methods of the present disclosure are useful for generating an animal model capable of displaying symptoms of Alzheimer's disease. Such models can then be used in the furtherance of therapeutics to treat Alzheimer's disease or other neurodegenerative disease. The present disclosure is related to a method of preparing a non-human animal model for Alzheimer's disease comprising administering to a non-human animal a miRNA compound that mimics miR-485-3p (“miRNA compound”). In some aspects, the present disclosure is related to a method of preparing a non-human animal model for Alzheimer's disease comprising administering to a non-human animal a miRNA compound that inhibit or reduce expression of a SIRT1 protein or a SIRT1 mRNA. In some aspects, the miRNA compound that inhibit or reduce expression of a SIRT1 protein or a SIRT1 mRNA is miR-485 or miR-485 mimic.

In some aspects, the miRNA compound is administered to the non-human animal (i) intrathecally or intracerebroventricularly at a dose of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, or at least about 1.0 mg/kg or (ii) parentherally, e.g., intravenously, at a dose of at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, at least about 20 mg/kg.

In some aspects, the miRNA compound is administered to the non-human animal about every 12 hours, about every 1 day, about every 2 days, about every 3 days, about every 4 days, about every 5 days, about every 6 days, about every 7 days, about a week, about two weeks, about three weeks, about four weeks, or about five weeks.

In some aspects, the miRNA compound is administered to the non-human animal for a duration of about 1 month, about 2 months, about 3 months, about 4 months, about 5 months, about 6 months, about 7 months, about 8 months, about 9 months, about 10 months, about 11 months, or about 1 year.

In some aspects, the miRNA compound is delivered in a delivery agent. In some aspects, the delivery agent is a micelle, an exosome, a lipid nanoparticle, an extracellular vesicle, or a synthetic vesicle. In some aspects, the miRNA compound is delivered by a viral vector. In some aspects, the viral vector is an AAV, an adenovirus, a retrovirus, or a lentivirus.

In some aspects, the viral vector is an AAV that has a serotype of AAV2, AAV3, AAV4, AAV5, AAV6, AAV7, AAV8, AAV9, AAV10, or any combination thereof. In some aspects, the serum level of the miRNA compound after the administration is at least about 10 nM, at least about 100 nM, at least about 1000 nM, or at least about 10,000 nM.

In some aspects, the serum level of the miRNA compound after the administration is between about 10 nM and about 10,000 nM, between about 20 nM and about 1000 nM, between about 30 nM and about 1000 nM, between about 40 nM and about 1000 nM, between about 50 nM and about 1000 nM, between about 60 nM and about 1000 nM, between about 70 nM and about 1000 nM, between about 80 nM and about 1000 nM, between about 90 nM and about 1000 nM, between about 100 nM and about 1000 nM, between about 200 nM and about 1000 nM, between about 300 nM and about 1000 nM, between about 400 nM and about 1000 nM, between about 500 nM and about 1000 nM, between about 600 nM and about 1000 nM, between about 700 nM and about 1000 nM, between about 800 nM and about 1000 nM, or between about 900 nM and about 1000 nM. In some aspects, the expression of the SIRT1 protein in the non-human animal model is reduced at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

The methods of the present disclosure are useful for generating a non-human animal model capable of displaying symptoms of Alzheimer's disease. Such models can then be used in the furtherance of therapeutics to treat Alzheimer's disease or other neurodegenerative disease. In some aspects, the non-human animal model exhibits one or more symptoms of Alzheimer's disease. In some aspects, the one or more symptoms of Alzheimer's disease are cognitive impairment and/or dementia. In some aspects, the non-human animal model shows one or more biochemical characteristics of Alzheimer's disease. In some aspects, one or more biochemical characteristics of Alzheimer's disease are (i) increased amyloid beta expression in CNS, (ii) increased tau expression in the CNS, (iii) increased amyloid plaques composed of amyloid-beta (Aβ) peptides, (iv) neurofibrillary tangles (NFTs) composed of hyperphosphorylated tau, or (v) any combination thereof. The non-human animal model for Alzheimer's disease of the present disclosure exhibits a higher level of miR-485-3p or its mimic in a biological sample, e.g., serum, saliva, urine, blood, cerebrospinal fluid, or any combination thereof.

### BRIEF DESCRIPTION OF DRAWINGS

FIG. 1A is a graph showing the analysis of miRNA expression patterns (volcano blot) in patient group versus control group; FIG. 1B shows analysis of miRNA expression patterns in patient group versus control group.

FIG. 2 is a list of 3′-untranslated region (UTR) of SIRT1 (SEQ ID NO: 43), and the miRNA binding portion (seed sequence) is shown (in the rectangle) from nucleotides 280-286.

FIG. 3A shows a portion of the 3′ UTR in an SIRT1 transcript (SEQ ID NO: 44) with the tgtatga seed sequence (bolded) (SEQ ID NO: 45). FIG. 3B shows the result of binding of miR-485-3p to SIRT1. Left two bars used wild type SIRT1 while the right two bars used mutated SIRT1.

FIG. 4A shows the expression of amyloid precursor protein (APP), and FIG. 4B shows the expression on truncated Tau and p-Tau after transfection of miR-485-3p mimic in Neuro 2a cells. Both figures compare the expression of the specific protein with the expression of beta-actin.

FIG. 5 shows the results of analysis of expression of SIRT1, c-fos (CFOS), APP and beta amyloid (Aβ) in the brain of normal mice intranasally treated with miR-485-3p mimic.

FIG. 6A shows the effect of administration of miR-485-3p mimic on production of Aβ 42 in wild type, wild type treated with miR-485-3p intranasally, and 5XFAD animal. FIG. 6B shows the production of Aβ oligomers in the hippocampus in wild type, wild type treated with miR-485-3p intranasally, and 5XFAD animal.

FIG. 7 shows the miR-485-3p sequence similarity between different species, i.e., Homo sapiens, Capra hircus, Ovis aries, Gorilla gorilla, Rattus norvegicus, Eptesicus fuscus, Mus musculus, Bos Taurus, Pongo pygmaeus, Macaca mulatta, Equus caballus, Pan troglodytes, Canis lupus familiaris, Oryctolagus cuniculus, Pan paniscus, Dasypus novemcinctus, and Pteropus Alecto.

FIG. 8 is a graph showing the results of comparison of cognitive functions of normal mice and 5xFAD and WT treated with miR-485-3p mimic intranasally.

FIGS. 9A and 9B show lentiviral injection sites and expression of miR-485-3p in mouse hippocampus. FIG. 9A shows a schematic diagram of sites in the mouse brain for lentivirus injection. 1* and 3* show injection sites in the dentate gyrus. 2* and 4* show injection sites in CA1. FIG. 9B shows lentiviral expression of miR-485-3p in the anterior and posterior hippocampus in the dentate gyrus and CA1.

FIGS. 10A-10D show the cognitive effects of miR-485-3p expression in the hippocampus on mice by the novel object recognition test. FIG. 10A shows the novel object recognition test experimental scheme. FIG. 10B shows the novelty preference and discrimination index for the novel object recognition test in miR-485-3p and control treated mice one hour after a 10 minute training. FIG. 10C shows the novelty preference and discrimination index for the novel object recognition test in miR-485-3p and control treated mice 24 hours after a 10 minute training. FIG. 10D shows the novelty preference and discrimination index for the novel object recognition test in miR-485-3p and control treated mice 3 weeks after a 10 minute training.

### DETAILED DESCRIPTION OF THE DISCLOSURE

The present disclosure is directed to a non-human animal model that exhibit one or more symptoms of Alzheimer's disease or methods of preparing the non-human animal model. The non-human animal model can be generated by injecting a miR485-3p or miR485-3p mimic.

### Terms

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C, A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “administration,” “administering,” and grammatical variants thereof refer to introducing a composition, such as a miRNA compound of the present disclosure, into a subject via a pharmaceutically acceptable route. The introduction of a composition, such as a miRNA compound of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some aspects, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

As used herein, the term “microRNAs,” “miRs,” or “miRNAs” refer to a class of short non-coding RNAs. miRNAs are regulators in almost all biological process. miRNAs are first transcribed as long transcripts and then processed by Drosha/DGCR8 and Dicer to a length of about 20-22 bases long. These RNAs are then loaded onto the RNA-induced silencing complex (RISC) to form mature gene-silencing complexes, which induce target mRNA degradation or transcription repression. Each miRNA targets hundreds of mRNAs, making miRNAs crucial regulators in the network of biological pathways. Because of the chemical nature of miRNAs, they can be synthesized, conjugated, locally or globally administrated, thus having a direct route toward therapeutic uses.

As used herein, the term “analog” or “mimic” when used in reference to microRNAs refers to any single or double stranded RNA oligonucleotides designed to supplement endogenous microRNA activity. Such an RNA fragment is designed to have its 5′-end bearing a partially complementary motif to the selected sequence in the 3′ UTR unique to the target gene. Once introduced into cells, this RNA fragment, mimicking an endogenous miRNA, can bind specifically to its target gene and produce posttranscriptional repression, more specifically translational inhibition, of the gene. Unlike endogenous miRNAs, miR-mimics act in a gene-specific fashion.

As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, that has undergone one or more processes of purification. In some aspects, isolating or purifying as used herein is the process of removing, partially removing (e.g., a fraction) of a composition of the present disclosure from a sample containing contaminants. In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some aspects can include one or more but preferably 6, 5, 4, 3, 2, or 1 mismatches with respect to the target pre-mRNA. Variations at any location within the oligomer are included. In certain aspects, antisense oligomers of the disclosure include variations in nucleobase sequence near the termini, variations in the interior, and if present are typically within about 6, 5, 4, 3, 2, or 1 subunits of the 5′ and/or 3′ terminus. In certain aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The term “non-human animal” as used herein refers to any non-human mammalian subject, including without limitation, domestic animals (e.g., dogs, cats and the like), farm animals (e.g., cows, sheep, pigs, horses and the like), and laboratory animals (e.g., monkey, rats, mice, rabbits, guinea pigs and the like). The “non-human animal” does not include human.

As used herein, the phrase “subject in need thereof” includes subjects, such as mammalian subjects, that would benefit from administration of an agent for treating Alzheimer's disease.

As used herein the term “therapeutically effective amount” is the amount of reagent or pharmaceutical compound comprising an agent for treating Alzheimer's disease that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The term “upstream” refers to a nucleotide sequence that is located 5′ to a reference nucleotide sequence.

The term “dose” refers to a quantity of a composition or drug taken at a particular administration. The term “dose” is an amount sufficient to induce one or more symptoms of Alzheimer's disease in a non-human animal. In some aspects, the dose can be expressed in mg/kg.

The term “dosing interval” refers to the period of time between two separate, adjacent administrations. In some aspects, the methods of the present disclosure dose not require any dosing interval, i.e., single administration. In other aspects, dosing Intervals between two dosages can be, for example, weekly, every 2 weeks, every 3 weeks, monthly, every three months or yearly. Intervals can also be irregular.

The term “dosing duration” or “duration” refers to the time period in which the miRNA composition of the present disclosure is administered according to the method disclosed herein. The duration of the present methods comprises the first and the last dosages and the dosing interval in between. For example, if the first dose is given on day 1, the second dose is given on day 8, the third dose is given on day 15, the fourth dose is given on day 22, and the fifth dose is given on day 29, the dosing duration is four weeks.

Various aspects of the disclosure are described in further detail in the following subsections.

### Non-Human Alzheimer's Disease Models

The non-human animal model of the present disclosure can be generated to display one or more symptoms of Alzheimer's disease. Such models can then be used in the furtherance of therapeutics to treat Alzheimer's disease or other neurodegenerative disease. The present disclosure is related to a non-human animal model for Alzheimer's disease (e.g., exhibiting one or more symptoms of Alzheimer's disease) by administering to a non-human animal a miRNA compound that mimics miR-485-3p (“miRNA compound”). In some aspects, the present disclosure is related to a non-human animal model for Alzheimer's disease by administering to a non-human animal a miRNA compound that inhibit or reduce expression of a SIRT1 protein or a SIRT1 mRNA. In some aspects, the miRNA compound that inhibit or reduce expression of a SIRT1 protein or a SIRT1 mRNA is miR-485 or miR-485 mimic. In some aspects, the non-human animal model for Alzheimer's disease of the present disclosure has a reduced expression of a SIRT1 protein or a SIRT1 mRNA. In other aspects, the non-human animal model for Alzheimer's disease of the present disclosure has an increased expression of amyloid beta. In some aspects, the non-human animal model for Alzheimer's disease of the present disclosure has an increased expression of phosphorylated Tau. In some aspects, the non-human animal model for Alzheimer's disease of the present disclosure has an increased expression of truncated Tau.

The non-human animal model for Alzheimer's disease of the present disclosure exhibits a higher level of miR-485-3p or its mimic in a biological sample, e.g., serum, saliva, urine, blood, cerebrospinal fluid, or any combination thereof, compared to the corresponding animal model that does not exhibit a symptom of Alzheimer's disease. In some aspects, the non-human animal model for Alzheimer's disease of the present disclosure exhibits at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 10.5 fold, at least about 11 fold, at least about 11.5 fold, at least about 12 fold, at least about 12.5 fold, at least about 13 fold, at least about 13.5 fold, at least about 14 fold, at least about 14.5 fold, at least about 15 fold, at least about 15.5 fold, at least about 16 fold, at least about 16.5 fold, at least about 17 fold, at least about 17.5 fold, at least about 18 fold, at least about 18.5 fold, at least about 19 fold, at least about 19.5 fold, or at least about 20 fold higher level of miR-485-3p or its mimic in a biological sample, e.g., serum, saliva, urine, blood, cerebrospinal fluid, or any combination thereof, compared to the corresponding animal model that does not exhibit a symptom of Alzheimer's disease.

The non-human animal model for Alzheimer's disease of the present disclosure exhibits at least about 21 fold, at least about 22 fold, at least about 23 fold, at least about 24 fold, at least about 25 fold, at least about 26 fold, at least about 27 fold, at least about 28 fold, at least about 29 fold, at least about 30 fold, at least about 31 fold, at least about 32 fold, at least about 33 fold, at least about 34 fold, at least about 35 fold, at least about 36 fold, at least about 37 fold, at least about 38 fold, at least about 39 fold, at least about 40 fold, at least about 45 fold, at least about 50 fold, at least about 55 fold, at least about 60 fold, at least about 65 fold, at least about 70 fold, at least about 75 fold, at least about 80 fold, at least about 85 fold, at least about 90 fold, at least about 95 fold, or at least about 100 fold higher level of miR-485-3p or its mimic in a biological sample, e.g., serum, saliva, urine, blood, cerebrospinal fluid, or any combination thereof, compared to the corresponding animal model that does not exhibit a symptom of Alzheimer's disease.

The non-human animal model for Alzheimer's disease of the present disclosure exhibits at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold, at least about 280 fold, at least about 290 fold, at least about 300 fold, at least about 310 fold, at least about 320 fold, at least about 330 fold, at least about 340 fold, at least about 350 fold, at least about 360 fold, at least about 370 fold, at least about 380 fold, at least about 390 fold, at least about 400 fold, at least about 500 fold, at least about 600 fold, at least about 700 fold, at least about 800 fold, at least about 9000 fold, at least about 1000 fold, at least about 1100 fold, or at least about 1200 fold higher level of miR-485-3p or its mimic in a biological sample, e.g., serum, saliva, urine, blood, cerebrospinal fluid, or any combination thereof, compared to the corresponding animal model that does not exhibit a symptom of Alzheimer's disease.

The higher level of the miRNA compound in the non-human animal model is not naturally occurring. In some aspects, the higher level of the miRNA compound is induced by an administration of the miRNA compound.

The miRNA compound of the present disclosure mimics the role of miR-485-3p, thereby exhibiting an effect of increasing expression or activation of miR-485-3p. In other aspects, the miRNA compound enhances the interaction of miR-485-3p with the 3′-UTR region of SIRT1 mRNA. In some aspects, the miRNA compound can be characterized by enhancing intracellular action or function of miR-485-3p. In the present disclosure, the miRNA compound can be an oligonucleotide which binds to all or a part of the nucleotide sequence UCAUACA (SEQ ID NO: 32) or GUCAUACACGGCUCUCCUCUCU (SEQ ID NO: 31). In some aspects, the RNA sequence disclosed herein can also be a DNA sequence; for example, GUCAUACACGGCUCUCCUCUCU (SEQ ID NO: 31) can be described as GTCATACACGGCTCTCCTCTCT (SEQ ID NO: 37).

In some aspects, the miRNA compound is an RNA sequence. In some aspects, the miRNA compound is a DNA sequence. In some aspects, the miRNA compound is a combination of an RNA and a DNA. In some aspects,

In some aspects, the miRNA compound can be selected from the group consisting of DNA, RNA, siRNA, shRNA, or any combination thereof. In some aspects, the miRNA compound includes DNA, RNA, polynucleotides, analogs and derivatives thereof. In some aspects, the miRNA compound is an arbitrary substance capable of mimicking the role of miR-485-3p, including a substance synthesizing the miR-485-3p sequence, small hairpin RNA molecules (shRNA), small interfering RNA molecules (siRNA), seeded target LNA (Locked Nucleic Acid) oligonucleotides, decoy oligonucleotides, an aptamer, a ribozyme, or an antibody recognizing a DNA: RNA hybrid.

In some aspects, the miRNA compound is an oligonucleotide capable of increasing the activity of miR-485-3p, including all or a part of the mature and/or mature sequence of miR-485-3p. Because miRNA binds to the target via the seed sequence, it can effectively inhibit translation of the target mRNA if the miRNA-485-3p interaction with the seed sequence is increased. In some aspects, the miRNA compound reduces the expression amount of SIRT1, increases the expression of c-Fos; induces the expression of amyloid precursor protein (APP) expression, and/or induces the production of Aβ or truncated Tau protein, and/or induces phosphorylation of Tau. In some aspects, the miRNA compound is administered to normal non-human animals and reduces the expression amount of SIRT1, increases the expression of CFOS, induces the expression of amyloid precursor protein (APP) expression, and/or induces the production of Aβ or truncated Tau protein, and/or induces phosphorylation of Tau.

In some aspects, the non-human animal can be any non-human animal, particularly a non-human mammal. Non-human mammals useful for the disclosure include, but are not limited to, domestic animals, farm animals, zoo animals such as bears, sport animals, pet animals such as dogs, cats, guinea pigs, rabbits, rats, mice, horses, cattle, bears, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; koalas, and so on. In certain aspects, the animal is a mouse. In other aspects, the animal is a chimpanzee. In some aspects, the animal is a rat.

In some aspects, the animal is goat (e.g., Capra hircus), sheep (e.g., Ovis aries), gorilla (e.g., Gorilla gorilla), rat (e.g., Rattus norvegicus), big brown bat (e.g., Eptesicus fuscus), mouse (e.g., Mus musculus), cattle (e.g., Bos taurus), Orangutan (Pongo pygmaeus), Rhesus monkey (e.g., Macaca mulatta), horse (e.g., Equus caballus), chimpanzee (e.g., Pan troglodytes), dog (e.g., Canis lupus familiaris), rabbit (e.g., Oryctolagus cuniculus), Pygmy chimpanzee (e.g., Pan paniscus), nine-banded armadillo (e.g, Dasypus novemcinctus), and black flying fox (e.g., apteropus Alecto).

In some aspects, a miRNA compound useful for the present method comprises a nucleotide sequence that is capable of binding to 5′ UCAUACA 3′ (SEQ ID NO: 32), wherein the miRNA compound comprises about 6 to about 30 nucleotides. The miRNA compound useful for the present disclosure is useful for reducing transcription and translation of SIRT1, a gene that is shows low expression in post-mortem analysis of brain tissue of Alzheimer's disease patients. In another aspect, the miRNA compound reduces transcription of an SIRT1 gene and/or expression of a SIRT1 protein.

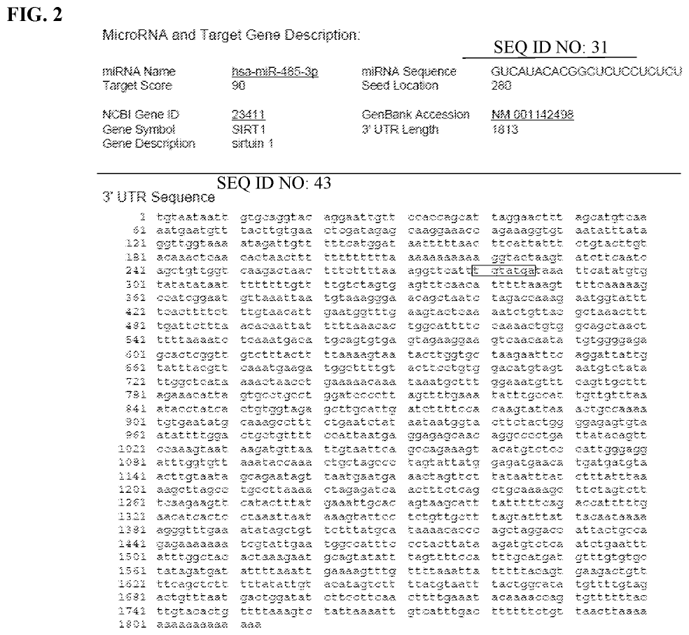

SIRT1 proteins are known as NAD-dependent protein deacetylase sirtuin-1, regulatory protein SIR2 homolog 1, SIR2-like protein 1, or hSIR2. The 3′ untranslated region of human SIRT1 protein (NM_001142498) is shown in FIG. 2. The corresponding 3′ UTR in other species, e.g., gorilla, chimpanzee, mouse, rat, goat, dog, big brown bat, sheep, and cattle, are shown in FIG. 7.

In some aspects, the miRNA compound comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides at the 5′ of the nucleotide sequence. In some aspects, the miRNA compound comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides at the 3′ of the nucleotide sequence.

In some aspects, the miRNA compound has a sequence selected from the group consisting of: GUCAUACA (SEQ ID NO: 1), UCAUACAC (SEQ ID NO: 2), UCAUACACG (SEQ ID NO: 3), UCAUACACGG (SEQ ID NO: 4), UCAUACACGGC (SEQ ID NO: 5), UCAUACACGGCU (SEQ ID NO: 6), UCAUACACGGCUC (SEQ ID NO: 7), UCAUACACGGCUCU (SEQ ID NO: 8), UCAUACACGGCUCUC (SEQ ID NO: 9), UCAUACACGGCUCUCC (SEQ ID NO: 10), UCAUACACGGCUCUCCU (SEQ ID NO: 11), UCAUACACGGCUCUCCUC (SEQ ID NO: 12), UCAUACACGGCUCUCCUCU (SEQ ID NO: 13), UCAUACACGGCUCUCCUCU (SEQ ID NO: 14), UCAUACACGGCUCUCCUCUC (SEQ ID NO: 15), UCAUACACGGCUCUCCUCUCU (SEQ ID NO: 16), GUCAUACAC (SEQ ID NO: 17), GUCAUACACG (SEQ ID NO: 18), GUCAUACACGG (SEQ ID NO: 19), GUCAUACACGGC (SEQ ID NO: 20), GUCAUACACGGCU (SEQ ID NO: 21), GUCAUACACGGCUC (SEQ ID NO: 22), GUCAUACACGGCUCU (SEQ ID NO: 23), GUCAUACACGGCUCUC (SEQ ID NO: 24), GUCAUACACGGCUCUCC (SEQ ID NO: 25), GUCAUACACGGCUCUCCU (SEQ ID NO: 26), GUCAUACACGGCUCUCCUC (SEQ ID NO: 27), GUCAUACACGGCUCUCCUCU (SEQ ID NO: 28), GUCAUACACGGCUCUCCUCUC (SEQ ID NO: 30), and GUCAUACACGGCUCUCCUCUCU (SEQ ID NO: 31), wherein U can optionally be T. In certain aspects, the miRNA compound comprises GUCAUACACGGCUCUCC (SEQ ID NO: 25). In some aspects, the miRNA compound comprises GTCATACACGGCTCTCC (SEQ ID NO: 38). In other aspects, the miRNA compound comprises GUCAUACACGGCUCUCCU (SEQ ID NO: 26). In some aspects, the miRNA compound comprises GTCATACACGGCTCTCCT (SEQ ID NO: 39). In some aspects, the miRNA compound comprises GUCAUACACGGCUCUCCUC (SEQ ID NO: 27). In some aspects, the miRNA compound comprises GTCATACACGGCTCTCCTC (SEQ ID NO: 40). In some aspects, the miRNA compound comprises GUCAUACACGGCUCUCCUCU (SEQ ID NO: 28). In some aspects, the miRNA compound comprises GTCATACACGGCTCTCCTCT (SEQ ID NO: 41). In some aspects, the miRNA compound comprises GUCAUACACGGCUCUCCUCUC (SEQ ID NO: 30). In some aspects, the miRNA compound comprises GTCATACACGGCTCTCCTCTC (SEQ ID NO: 42). In some aspects, the miRNA compound comprises GUCAUACACGGCUCUCCUCUCU (SEQ ID NO: 31). In some aspects, the miRNA compound comprises GTCATACACGGCTCTCCTCTCT (SEQ ID NO: 37).

In some aspects, the sequence of the miRNA compound is at least about 50%, at least about 55%, at least about 60%, at least about 65%, at least about 70%, at least about 75%, at least about 80%, at least about 85%, at least about 90%, or at least about 95% sequence identity to GUCAUACACGGCUCUCCUCUCU (SEQ ID NO: 31), wherein U can be optionally T. In some aspects, the miRNA compound has a sequence that has at least about 90% sequence identity to GUCAUACACGGCUCUCCUCUCU (SEQ ID NO: 31). In some aspects, the miRNA compound as the sequence as set forth in 5′ UCAUACA 3′, wherein the miRNA compound is at least about 80%, at least about 90%, or at least about 95% identical to GUCAUACACGGCUCUCCUCUCU (SEQ ID NO: 31). In some aspects, the miRNA compound comprises the nucleotide sequence GUCAUACACGGCUCUCCUCUCU (SEQ ID NO: 31) with one mismatch or two mismatches. In some aspects, the miRNA compound useful for the disclosure comprises the nucleotide sequence GUCAUACACGGCUCUCCUCUCU (SEQ ID NO: 31).

In some aspects, the miRNA compound can be characterized in that it comprises at least one chemical modification at one or more nucleotides. The chemical modification can be for enhancing in vivo stability, imparting nucleic acid degrading enzyme resistance, and/or reducing nonspecific immune response. In some aspects, the miRNA compound comprises at least one modified nucleotide. In some aspects, the at least one modified nucleotide is a locked nucleic acid (LNA), an unlocked nucleic acid (UNA), a bridged nucleic acid (BNA), an arabino nucleic acid (ANA), and/or a peptide nucleic acid (PNA). In some aspects, the miRNA compound comprises a backbone modification. In some aspects, the backbone modification is a phosphorodiamidate morpholino oligomer (PMO) or phosphorothioate modification.

In some aspects, the chemical modification can be at the 2′ carbon position of the sugar structure in the nucleotide, the hydroxyl group (—OH) is substituted with a methyl group (—CH3), a methoxy group (—OCH3), an amine group (—NH2), O-2-methoxyethyl group, O-propyl group, O-2-methylthioethyl group, O-3-aminopropyl group, O-3-dimethylaminopropyl group, or an ethyl group. In other aspects, the chemical modification can be at the oxygen of the sugar structure wherein the nucleotide is replaced by sulfur. In other aspects, the chemical modification can be one or more modifications selected from the group consisting of a phosphorothioate bond, a boranophosphate bond, and a methyl phosphonate bond.

In some aspects, a miRNA compound of the present disclosure is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a miRNA compound of the present disclosure can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In another aspect, the miRNA compound of the present disclosure can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the miRNA compound (e.g., an miR485 mimic) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide (e.g., an miR485 mimic) are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### 1. Base Modifications

In certain aspects, the chemical modification is at nucleobases in a miRNA compound of the present disclosure (e.g., an miR485 mimic). In some aspects, the at least one chemically modified nucleoside is a modified uridine (e.g., pseudouridine (Ψ), 2-thiouridine (s2U), 1-methyl-pseudouridine (m1Ψ), 1-ethyl-pseudouridine (e1Ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the miRNA compound of the present disclosure (e.g., an miR485 mimic) is uniformly modified (e.g., fully modified, modified throughout the entire sequence) for a particular modification. For example, a miRNA compound can be uniformly modified with the same type of base modification, e.g., 5-methyl-cytidine (m5C), meaning that all cytosine residues in the miRNA compound sequence are replaced with 5-methyl-cytidine (m5C). Similarly, a miRNA compound can be uniformly modified for any type of nucleoside residue present in the sequence by replacement with a modified nucleoside such as any of those set forth above.

In some aspects, the miRNA compound of the present disclosure (e.g., an miR485 mimic) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., an miR485 mimic) are modified nucleobases.

### 2. Backbone Modifications

In some aspects, the payload can comprise a “miRNA compound of the present disclosure” (for example comprising an miR485 mimic), wherein the miRNA compound includes any useful modification to the linkages between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

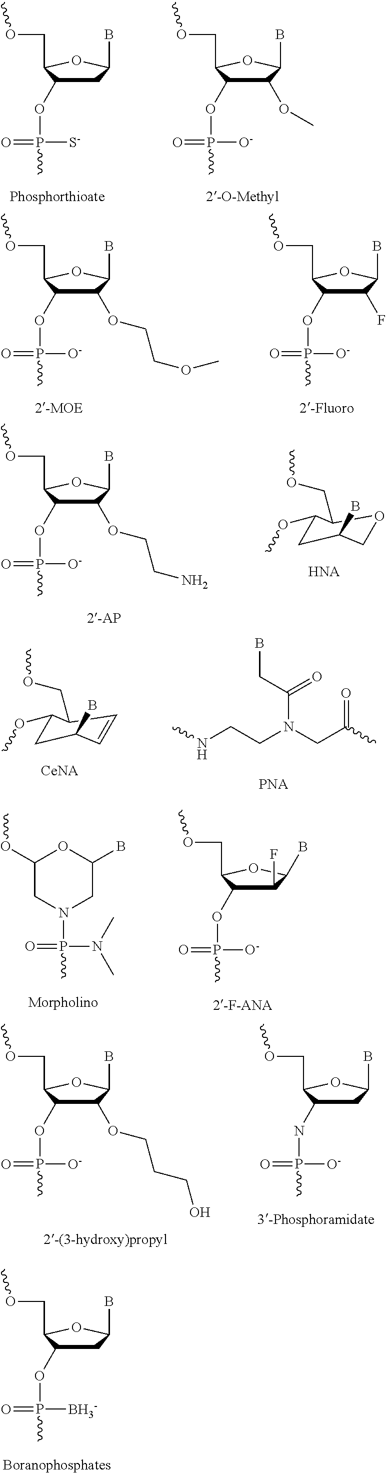

In some aspects, the presence of a backbone linkage disclosed above increase the stability (e.g., thermal stability) and/or resistance to degradation (e.g., enzyme degradation) of a polynucleotide of the present disclosure (e.g., an miR485 mimic). In some aspects, the stability and/or resistance to degradation increases by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in the modified miRNA compound compared to a corresponding miRNA compound without the modification (reference or control polynucleotide).

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a miRNA compound of the present disclosure (e.g., an miR485 mimic) are modified (e.g., all of them are phosphorothioate).

In some aspects, 1, 2, 3, 4, 5, 6, 7, 8, 9, 10, 11, 12, 13, 14, 15, 16, 17, 18, 19, 20, or 21 backbone linkages in a miRNA compound of the present disclosure (e.g., an miR485 mimic) are modified (e.g., phosphorothioate).

In some aspects, the backbone comprises linkages selected from the group consisting of phosphodiester linkage, phosphotriesters linkage, methylphosphonate linkage, phosphoramidate linkage, phosphorothioate linkage, and combinations thereof.

### 3. Sugar Modifications

The modified nucleosides and nucleotides which can be incorporated into a miRNA compound of the present disclosure (e.g., an miR485 mimic), can be modified on the sugar of the nucleic acid. Thus, in some aspects, the payload comprises a nucleic acid, wherein the nucleic comprises at least one nucleoside analog (e.g., a nucleoside with a sugar modification).

In some aspects, the sugar modification increases the affinity of the binding of a MBS to its target miRNA. Incorporating affinity-enhancing nucleotide analogues in the MBS, such as LNA or 2′-substituted sugars can allow the length of MBS to be reduced, and also may reduce the upper limit of the size an MBS before non-specific or aberrant binding takes place.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the nucleotides in a miRNA compound of the present disclosure (e.g., an miR485 mimic) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a miRNA compound of the present disclosure (e.g., an miR485 mimic) are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleoside analogues present in a miRNA compound of the present disclosure (e.g., an miR485 antimir) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. See, e.g., International Publ. Appl. No.

In some aspects, nucleoside analogs present in a miRNA compound of the present disclosure comprise Locked Nucleic Acid (LNA); 2′-0-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-0-methyl nucleic acid (2′-OMe), 2′-0-methoxyethyl nucleic acid (2′-MOE), or any combination thereof.

In some aspects, a miRNA compound of the present disclosure (e.g., an miR485 mimic) can comprise both modified RNA nucleotide analogues (e.g., LNA) and DNA units. In some aspects, a miRNA compound of the present disclosure is a gapmer. In some aspects, a miRNA compound is a mixmer.

### Methods of Preparing Alzheimer Disease Model

The methods of the present disclosure are useful for generating an animal model capable of displaying symptoms of Alzheimer's disease. Such models can then be used in the furtherance of therapeutics to treat Alzheimer's disease or other neurodegenerative disease. The present disclosure is related to a method of preparing a non-human animal model for Alzheimer's disease comprising administering to a non-human animal a miRNA compound that mimics miR-485-3p (“miRNA compound”). In some aspects, the present disclosure is related to a method of preparing a non-human animal model for Alzheimer's disease comprising administering to a non-human animal a miRNA compound that inhibit or reduce expression of a SIRT1 protein or a SIRT1 mRNA. In some aspects, the miRNA compound that inhibit or reduce expression of a SIRT1 protein or a SIRT1 mRNA is miR-485 or miR-485 mimic.

In some aspects, the miRNA compound is administered, e.g., parenterally. In some aspects, the miRNA compound is administered intravenously, to the non-human animal. In some aspects, the parentheral, e.g., intravenous, administration is at a dose of at least about 0.1 mg/kg, at least about 0.5 mg/kg, at least about 1.0 mg/kg, at least about 1.5 mg/kg, at least about 2.0 mg/kg, at least about 2.5 mg/kg, at least about 3.0 mg/kg, at least about 3.5 mg/kg, at least about 4.0 mg/kg, at least about 4.5 mg/kg, at least about 5.0 mg/kg, at least about 5.5 mg/kg, at least about 6.0 mg/kg, at least about 6.5 mg/kg, at least about 7.0 mg/kg, at least about 7.5 mg/kg, at least about 8.0 mg/kg, at least about 8.5 mg/kg, at least about 9.0 mg/kg, at least about 9.5 mg/kg, at least about 10.0 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, at least about 20 mg/kg, at least about 21 mg/kg, at least about 22 mg/kg, at least about 23 mg/kg, at least about 24 mg/kg, at least about 25 mg/kg, at least about 26 mg/kg, at least about 27 mg/kg, at least about 28 mg/kg, at least about 29 mg/kg, at least about 30 mg/kg, at least about 31 mg/kg, at least about 32 mg/kg, at least about 33 mg/kg, at least about 34 mg/kg, at least about 35 mg/kg, at least about 36 mg/kg, at least about 37 mg/kg, at least about 38 mg/kg, at least about 39 mg/kg, or at least about 40 mg/kg, for a dosing interval disclosed herein, e.g., once a week for a month. In some aspects, the miRNA is administered, e.g., parenterally, e.g., intravenously, to the non-human animal at least about 6 mg/kg, at least about 7 mg/kg, at least about 8 mg/kg, at least about 9 mg/kg, or at least about 10 mg/kg, for a dosing interval disclosed herein, e.g., once a week for a month. In some aspects, the miRNA is administered, e.g., parenterally, e.g., intravenously, to the non-human animal at least about 10 mg/kg, at least about 12 mg/kg, at least about 14 mg/kg, at least about 16 mg/kg, at least about 18 mg/kg, or at least about 20 mg/kg, for a dosing interval disclosed herein, e.g., once a week for a month.

In other aspects, the miRNA is administered, e.g., parenterally, e.g., intravenously, to the non-human animal between about 1 mg/kg and about 100 mg/kg, between about 1 mg/kg and about 90 mg/kg, between about 1 mg/kg and about 80 mg/kg, between about 1 mg/kg and about 70 mg/kg, between about 1 mg/kg and about 60 mg/kg, between about 1 mg/kg and about 50 mg/kg, between about 1 mg/kg and about 40 mg/kg, between about 1 mg/kg and about 30 mg/kg, between about 1 mg/kg and about 20 mg/kg, between about 1 mg/kg and about 10 mg/kg, between about 5 mg/kg and about 50 mg/kg, between about 5 mg/kg and about 40 mg/kg, between about 5 mg/kg and about 30 mg/kg, between about 5 mg/kg and about 20 mg/kg, between about 10 mg/kg and about 50 mg/kg, between about 10 mg/kg and about 40 mg/kg, between about 10 mg/kg and about 30 mg/kg, between about 10 mg/kg and about 20 mg/kg, or between about 15 mg/kg and about 20 mg/kg, for a dosing interval disclosed herein, e.g., once a week for a month.

In other aspects, the miRNA is administered, e.g., parenterally, e.g., intravenously, to the non-human animal between about 1 mg/kg and about 3 mg/kg, e.g., about 2 mg/kg, between about 3 mg/kg and about 5 mg/kg, e.g., about 4 mg/kg, between about 9 mg/kg and about 11 mg/kg, e.g., about 10 mg/kg, or between about 19 mg/kg and about 21 mg/kg, e.g., about 20 mg/kg. In other aspects, the miRNA is administered, e.g., parenterally, e.g., intravenously, to the non-human animal between about 1 mg/kg and about 3 mg/kg, e.g., about 1 mg/kg, about 2 mg/kg, or about 3 mg/kg, between about 3 mg/kg and about 5 mg/kg, e.g., about 3 mg/kg, about 4 mg/kg, or about 5 mg/kg, between about 5 mg/kg and about 7 mg/kg, about 5 mg/kg, about 6 mg/kg, about 7 mg/kg, between about 7 mg/kg and about 9 mg/kg, about 7 mg/kg, about 8 mg/kg, or about 9 mg/kg, between about 9 mg/kg and about 11 mg/kg, about 9 mg/kg, about 10 mg/kg, or about 11 mg/kg, between about 11 mg/kg and about 13 mg/kg, about 11 mg/kg, about 12 mg/kg, or about 13 mg/kg, between about 13 mg/kg and about 15 mg/kg, about 13 mg/kg, about 14 mg/kg, or about 15 mg/kg, between about 15 mg/kg and about 17 mg/kg, about 15 mg/kg, about 16 mg/kg, about 17 mg/kg, between about 17 mg/kg and about 19 mg/kg, about 17 mg/kg, about 18 mg/kg, or about 19 mg/kg, or between about 19 mg/kg and about 21 mg/kg, about 19 mg/kg, about 20 mg/kg, or about 21 mg/kg. In other aspects, the miRNA is administered, e.g., parenterally, e.g., intravenously, to the non-human animal at a dose of about 2 mg/kg once a week for 1 month. In other aspects, the miRNA is administered, e.g., parenterally, e.g., intravenously, to the non-human animal at a dose of about 4 mg/kg once a week for 1 month. In other aspects, the miRNA is administered, e.g., parenterally, e.g., intravenously, to the non-human animal at a dose of about 10 mg/kg once a week for 1 month. In other aspects, the miRNA is administered, e.g., parenterally, e.g., intravenously, to the non-human animal at a dose of about 20 mg/kg, for a dosing interval disclosed herein, e.g., once a week for 1 month.

In some aspects, the miRNA compound is administered, e.g., intrathecally or intracerebroventricularly, to the non-human animal at a dose of at least about 0.05 mg/kg, at least about 0.06 mg/kg, at least about 0.07 mg/kg, at least about 0.08 mg/kg, at least about 0.09 mg/kg, at least about 0.1 mg/kg, at least about 0.11 mg/kg, at least about 0.12 mg/kg, at least about 0.13 mg/kg, at least about 0.14 mg/kg, at least about 0.15 mg/kg, at least about 0.16 mg/kg, at least about 0.17 mg/kg, at least about 0.18 mg/kg, at least about 0.19 mg/kg, at least about 0.2 mg/kg, at least about 0.21 mg/kg, at least about 0.22 mg/kg, at least about 0.23 mg/kg, at least about 0.24 mg/kg, at least about 0.25 mg/kg, at least about 0.26 mg/kg, at least about 0.27 mg/kg, at least about 0.28 mg/kg, at least about 0.29 mg/kg, at least about 0.3 mg/kg, at least about 0.31 mg/kg, at least about 0.32 mg/kg, at least about 0.33 mg/kg, at least about 0.34 mg/kg, at least about 0.35 mg/kg, at least about 0.36 mg/kg, at least about 0.37 mg/kg, at least about 0.38 mg/kg, at least about 0.39 mg/kg, at least about 0.4 mg/kg, at least about 0.41 mg/kg, at least about 0.42 mg/kg, at least about 0.43 mg/kg, at least about 0.44 mg/kg, at least about 0.45 mg/kg, at least about 0.46 mg/kg, at least about 0.47 mg/kg, at least about 0.48 mg/kg, at least about 0.49 mg/kg, at least about 0.5 mg/kg, at least about 0.51 mg/kg, at least about 0.52 mg/kg, at least about 0.53 mg/kg, at least about 0.54 mg/kg, at least about 0.55 mg/kg, at least about 0.56 mg/kg, at least about 0.57 mg/kg, at least about 0.58 mg/kg, at least about 0.59 mg/kg, at least about 0.6 mg/kg, at least about 0.61 mg/kg, at least about 0.62 mg/kg, at least about 0.63 mg/kg, at least about 0.64 mg/kg, at least about 0.65 mg/kg, at least about 0.66 mg/kg, at least about 0.67 mg/kg, at least about 0.68 mg/kg, at least about 0.69 mg/kg, at least about 0.7 mg/kg, at least about 0.71 mg/kg, at least about 0.72 mg/kg, at least about 0.73 mg/kg, at least about 0.74 mg/kg, at least about 0.75 mg/kg, at least about 0.76 mg/kg, at least about 0.77 mg/kg, at least about 0.78 mg/kg, at least about 0.79 mg/kg, at least about 0.8 mg/kg, at least about 0.81 mg/kg, at least about 0.82 mg/kg, at least about 0.83 mg/kg, at least about 0.84 mg/kg, at least about 0.85 mg/kg, at least about 0.86 mg/kg, at least about 0.87 mg/kg, at least about 0.88 mg/kg, at least about 0.89 mg/kg, at least about 0.9 mg/kg, at least about 0.91 mg/kg, at least about 0.92 mg/kg, at least about 0.93 mg/kg, at least about 0.94 mg/kg, at least about 0.95 mg/kg, at least about 0.96 mg/kg, at least about 0.97 mg/kg, at least about 0.98 mg/kg, at least about 0.99 mg/kg, or at least about 1 mg/kg, at a dosing interval disclosed herein, e.g., once a week for a month.

In some aspects, the miRNA compound is administered, e.g., intrathecally or intracerebroventricularly, to the non-human animal at a dose between about 0.05 mg/kg and about 2 mg/kg, between about 0.05 mg/kg and about 1.5 mg/kg, between about 0.05 mg/kg and about 1 mg/kg, between about 0.06 mg/kg and about 2 mg/kg, between about 0.06 mg/kg and about 1.5 mg/kg, between about 0.06 mg/kg and about 1 mg/kg, between about 0.07 mg/kg and about 2 mg/kg, between about 0.07 mg/kg and about 1.5 mg/kg, between about 0.07 mg/kg and about 1 mg/kg, between about 0.02 mg/kg and about 2 mg/kg, between about 0.02 mg/kg and about 1.5 mg/kg, between about 0.02 mg/kg and about 1 mg/kg, between about 0.03 mg/kg and about 2 mg/kg, between about 0.03 mg/kg and about 1.5 mg/kg, between about 0.03 mg/kg and about 1 mg/kg, between about 0.04 mg/kg and about 2 mg/kg, between about 0.04 mg/kg and about 1.5 mg/kg, or between about 0.04 mg/kg and about 1 mg/kg at a dosing interval disclosed herein, e.g., once a week for a month.

In some aspects, the miRNA compound is administered, e.g., intrathecally or intracerebroventricularly, to the non-human animal at a dose between about 0.07 mg/kg and about 0.08 mg/kg, e.g., 0.07 mg/kg, 0.075 mg/kg, or about 0.08 mg/kg, between about 0.14 mg/kg and about 0.16 mg/kg, e.g., about 0.14 mg/kg, about 0.15 mg/kg, or about 0.16 mg/kg, between about 0.34 mg/kg and about 0.36 mg/kg, e.g., about 0.34 mg/kg, about 0.35 mg/kg, or about 0.36 mg/kg, or between 0.74 mg/kg and about 0.76 mg/kg, e.g., about 0.74 mg/kg, about 0.75 mg/kg, or about 0.76 mg/kg, at a dosing interval disclosed herein, e.g., once a week for a month.

In some aspects, the miRNA compound is administered, e.g., intrathecally or intracerebroventricularly, to the non-human animal at a dose of 0.075 mg/kg at a dosing interval disclosed herein, e.g., once a week for a month. In some aspects, the miRNA compound is administered, e.g., intrathecally or intracerebroventricularly, to the non-human animal at a dose of 0.15 mg/kg at a dosing interval disclosed herein, e.g., once a week for a month. In some aspects, the miRNA compound is administered, e.g., intrathecally or intracerebroventricularly, to the non-human animal at a dose of 0.35 mg/kg at a dosing interval disclosed herein, e.g., once a week for a month. In some aspects, the miRNA compound is administered, e.g., intrathecally or intracerebroventricularly, to the non-human animal at a dose of 0.75 mg/kg at a dosing interval disclosed herein, e.g., once a week for a month.

In some aspects, the miRNA compound is administered to the non-human animal at a dosing interval of about a week, about two weeks, about three weeks, about four weeks, about a month, about five weeks, about six weeks, about seven weeks, about eight weeks, about nine weeks, about ten weeks, about eleven weeks, or about twelve weeks. In some aspects, the miRNA compound is administered to the non-human animal at a duration of about thirteen weeks, about fourteen weeks, about 15 weeks, about 16 weeks, about 17 weeks, about 18 weeks, about 19 weeks, about 20 weeks, about 2 months, about 3 months, about 4 months, about 5 months, about 6 months, about 7 months, about 8 months, about 9 months, about 10 months, about 11 months, or about 12 months.

In some aspects, the miRNA compound is administered to the non-human animal at a dosing interval of about 12 hours, about 1 day, about 2 days, about 3 days, about 4 days, about 5 days, about 6 days, or about 7 days. In some In some aspects, the miRNA is administered to the non-human animal at a duration of about 1 month, about 2 months, about 3 months, about 4 months, about 5 months, about 6 months, about 7 months, about 8 months, about 9 months, about 10 months, about 11 months, or about 1 year. In some aspects, the miRNA compound is administered to the non-human animal once a week. In some aspects, the miRNA compound is administered to the non-human animal once a week for a duration of a month.

In some aspects, the miRNA compound is delivered in a delivery agent. In some aspects, the delivery agent is a micelle, an exosome, a lipid nanoparticle, an extracellular vesicle, or a synthetic vesicle. In some aspects, the miRNA compound is delivered by a viral vector. In some aspect s, the miRNA compound is delivered in a micelle.

In some aspects, the viral vector is an AAV, an adenovirus, a retrovirus, or a lentivirus. In some aspects, the viral vector is an AAV that has a serotype selected from the group consisting of AAV2, AAV3, AAV4, AAV5, AAV6, AAV7, AAV8, AAV9, or AAV10. In some aspects, the viral vector is HIV. In other aspects, the viral vector is a lentiviral vector.

In some aspects, the serum level of the miRNA compound after the administration is at least about 10 nM, at least about 100 nM, at least about 1000 nM, or at least about 10,000 nM. In some aspects, the serum level of the miRNA compound after the administration is at least about 10 nM, at least about 20 nM, at least about 30 nM, at least about 40 nM, at least about 50 nM, at least about 60 nM, at least about 70 nM, at least about 80 nM, at least about 90 nM, or at least about 100 nM. In some aspects, the serum level of the miRNA compound after the administration is at least about 100 nM, at least about 110 nM, at least about 120 nM, at least about 130 nM, at least about 140 nM, at least about 150 nM, at least about 160 nM, at least about 170 nM, at least about 180 nM, at least about 190 nM, or at least about 200 nM. In some aspects, the serum level of the miRNA compound after the administration is at least about 210 nM, at least about 220 nM, at least about 230 nM, at least about 240 nM, at least about 250 nM, at least about 260 nM, at least about 270 nM, at least about 280 nM, at least about 290 nM, or at least about 300 nM. In some aspects, the serum level of the miRNA compound after the administration is at least about 310 nM, at least about 320 nM, at least about 330 nM, at least about 340 nM, at least about 350 nM, at least about 360 nM, at least about 370 nM, at least about 380 nM, at least about 390 nM, or at least about 400 nM. In some aspects, the serum level of the miRNA compound after the administration is at least about 410 nM, at least about 420 nM, at least about 430 nM, at least about 440 nM, at least about 450 nM, at least about 460 nM, at least about 470 nM, at least about 480 nM, at least about 490 nM, or at least about 500 nM. In some aspects, the serum level of the miRNA compound after the administration is at least about 510 nM, at least about 520 nM, at least about 530 nM, at least about 540 nM, at least about 550 nM, at least about 560 nM, at least about 570 nM, at least about 580 nM, at least about 590 nM, or at least about 600 nM. In some aspects, the serum level of the miRNA compound after the administration is at least about 610 nM, at least about 620 nM, at least about 630 nM, at least about 640 nM, at least about 650 nM, at least about 660 nM, at least about 670 nM, at least about 680 nM, at least about 690 nM, or at least about 700 nM. In some aspects, the serum level of the miRNA compound after the administration is at least about 710 nM, at least about 720 nM, at least about 730 nM, at least about 740 nM, at least about 750 nM, at least about 760 nM, at least about 770 nM, at least about 780 nM, at least about 790 nM, or at least about 800 nM. In some aspects, the serum level of the miRNA compound after the administration is at least about 810 nM, at least about 820 nM, at least about 830 nM, at least about 840 nM, at least about 850 nM, at least about 860 nM, at least about 870 nM, at least about 880 nM, at least about 890 nM, or at least about 900 nM. In some aspects, the serum level of the miRNA compound after the administration is at least about 910 nM, at least about 920 nM, at least about 930 nM, at least about 940 nM, at least about 950 nM, at least about 960 nM, at least about 970 nM, at least about 980 nM, at least about 990 nM, or at least about 1000 nM.

In some aspects, the serum level of the miRNA compound after the administration is between about 10 nM and about 50 nM, between about 20 nM and about 100 nM, between about 30 nM and about 100 nM, between about 40 nM and about 100 nM, between about 50 nM and about 100 nM, between about 50 nM and about 200 nM, between about 50 nM and about 250 nM, between about 50 nM and about 300 nM, between about 50 nM and about 350 nM, between about 50 nM and about 400 nM, between about 50 nM and about 450 nM, or between about 50 nM and about 500 nM. In some aspects, the serum level of the miRNA compound after the administration is between about 100 nM and about 200 nM, between about 100 nM and about 300 nM, between about 100 nM and about 400 nM, between about 100 nM and about 500 nM, between about 100 nM and about 600 nM, between about 100 nM and about 700 nM, between about 100 nM and about 800 nM, between about 100 nM and about 900 nM, between about 100 nM and about 1000 nM, between about 200 nM and about 300 nM, between about 200 nM and about 400 nM, between about 200 nM and about 500 nM, between about 300 nM and about 400 nM, between about 300 nM and about 500 nM, between about 300 nM and about 600 nM, between about 300 nM and about 700 nM, between about 400 nM and about 500 nM, between about 400 nM and about 600 nM, between about 400 nM and about 700 nM, between about 500 nM and about 600 nM, between about 500 nM and about 700 nM, between about 500 nM and about 800 nM, between about 500 nM and about 900 nM, or between about 500 nM and about 1000 nM.

In some aspects, the serum level of the miRNA compound after the administration is between about 10 nM and about 10,000 nM, between about 20 nM and about 1000 nM, between about 30 nM and about 1000 nM, between about 40 nM and about 1000 nM, between about 50 nM and about 1000 nM, between about 60 nM and about 1000 nM, between about 70 nM and about 1000 nM, between about 80 nM and about 1000 nM, between about 90 nM and about 1000 nM, between about 100 nM and about 1000 nM, between about 200 nM and about 1000 nM, between about 300 nM and about 1000 nM, between about 400 nM and about 1000 nM, between about 500 nM and about 1000 nM, between about 600 nM and about 1000 nM, between about 700 nM and about 1000 nM, between about 800 nM and about 1000 nM, or between about 900 nM and about 1000 nM.

In some aspects, the expression of the SIRT1 protein is reduced at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some aspects, the non-human animal model no longer expresses any detectable level of the SIRT1 protein.

The methods of the present disclosure are useful for generating a non-human animal model capable of displaying symptoms of Alzheimer's disease. Such models can then be used in the furtherance of therapeutics to treat Alzheimer's disease or other neurodegenerative disease. In some aspects, the non-human animal model exhibits one or more symptoms of Alzheimer's disease. In some aspects, the one or more symptoms of Alzheimer's disease are selected from the group consisting of cognitive impairment and dementia. In some aspects, the non-human animal model shows one or more biochemical characteristics of Alzheimer's disease. In some aspects, the non-human animal model shows one or more biochemical characteristics of Alzheimer's disease are selected from the group consisting of: (i) increased amyloid beta expression in CNS, (ii) increased tau expression in CNS, (iii) increased amyloid plaques composed of amyloid-beta (Aβ) peptides, (iv) and/or neurofibrillary tangles (NFTs) composed of hyperphosphorylated tau. In some aspects, the non-human animal model shows one or more functional characteristics of Alzheimer's disease. In some aspects, the functional characteristics are selected from the group consisting of: difficulty completing tasks, repetitive behaviors, or decreased motor skills.

The method of the present disclosure are useful to screen for potential agents and therapeutics for use in treatment of Alzheimer's disease or other neurodegenerative disease. In some aspects, the methods of the present disclosure comprise screening an agent for its therapeutic effect in treating Alzheimer's disease in the non-human animal model. In some aspects, the agent is capable of reducing one or more symptoms of Alzheimer's disease. In some aspects, the one or more symptoms of Alzheimer's disease are selected from the group consisting of cognitive impairment and dementia. In some aspects, the agent is capable of improving one or more biochemical characteristics of Alzheimer's disease in a subject. In some aspects, the one or more biochemical characteristics of Alzheimer's disease are (i) increased amyloid beta expression in CNS, (ii) increased tau expression in CNS, (iii) increased amyloid plaques composed of amyloid-beta (Aβ) peptides, (iv) neurofibrillary tangles (NFTs) composed of hyperphosphorylated tau, or (v) any combination thereof. In some aspects, the agent is capable of improving one or more functional characteristics of Alzheimer's disease in a subject. In some aspects, the one or more functional characteristics of Alzheimer's disease are difficulty completing tasks, repetitive behaviors, decreased motor skills, or any combination thereof.

The methods of the present disclosure are useful for identifying agents that increase the transcription of the SIRT1 gene and/or increase the expression of the SIRT1 protein in a subject. In some aspects, the agent is capable of increasing the transcription of the SIRT1 gene and/or expression of the SIRT1 protein in a subject. In some aspects, the transcription of the SIRT1 gene or the expression of the SIRT1 protein is increased after administration of the agent by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

The compositions and methods of the present disclosure are also useful for treating Alzheimer's disease, comprising administering a candidate agent to the animal model and evaluating the therapeutic effect of the candidate agent on Alzheimer's disease by observing the degree of improvement of the symptoms of Alzheimer's disease. Using the compositions and methods disclosed herein, the candidate agent can a newly synthesized or known compound, and includes, without limitation, agents expected to have an effect on the prevention or treatment of Alzheimer's disease. In some aspects, the method further comprises administering a therapeutically effective amount of the agent to a subject. In some aspects, the evaluation of the therapeutic effect of Alzheimer's disease can include analysis of the interaction between miR-485-3p and the 3′-UTR of SIRT1 mRNA; analysis of expression of Aβ 42, analysis of the expression of amyloid precursor protein (APP); and/or analysis of the phosphorylation of Tau. These analysis techniques can be performed via Northern blotting, RT-PCR, hybridization assays, and similar techniques known to one of skill in the art.

In some aspects, the transcription of the SIRT1 gene and/or expression of the SIRT1 protein in the non-human animal model is reduced by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some aspects, the subject no longer exhibits the one or more symptoms of Alzheimer's disease.

The methods of the present disclosure are useful for generating a mouse model that is capable of displaying characteristics and/or symptoms of Alzheimer's Disease. In some aspects, the model is a non-human animal model for Alzheimer's disease comprising a miRNA compound comprising a nucleotide sequence comprising 5′ UCAUACA 3′ (SEQ ID NO: 32) wherein the nucleotide sequence comprises about 6 to about 30 nucleotides, and wherein the miRNA compound reduces transcription of an SIRT1 gene and/or expression of a SIRT1 protein. In some aspects, the miRNA compound has a sequence selected from the group consisting of: GUCAUACA (SEQ ID NO: 1), UCAUACAC (SEQ ID NO: 2), UCAUACACG (SEQ ID NO: 3), UCAUACACGG (SEQ ID NO: 4), UCAUACACGGC (SEQ ID NO: 5), UCAUACACGGCU (SEQ ID NO: 6), UCAUACACGGCUC (SEQ ID NO: 7), UCAUACACGGCUCU (SEQ ID NO: 8), UCAUACACGGCUCUC (SEQ ID NO: 9), UCAUACACGGCUCUCC (SEQ ID NO: 10), UCAUACACGGCUCUCCU (SEQ ID NO: 11), UCAUACACGGCUCUCCUC (SEQ ID NO: 12), UCAUACACGGCUCUCCUCU (SEQ ID NO: 13), UCAUACACGGCUCUCCUCU (SEQ ID NO: 14), UCAUACACGGCUCUCCUCUC (SEQ ID NO: 15), UCAUACACGGCUCUCCUCUCU (SEQ ID NO: 16), GUCAUACAC (SEQ ID NO: 17), GUCAUACACG (SEQ ID NO: 18), GUCAUACACGG (SEQ ID NO: 19), GUCAUACACGGC (SEQ ID NO: 20), GUCAUACACGGCU (SEQ ID NO: 21), GUCAUACACGGCUC (SEQ ID NO: 22), GUCAUACACGGCUCU (SEQ ID NO: 23), GUCAUACACGGCUCUC (SEQ ID NO: 24), GUCAUACACGGCUCUCC (SEQ ID NO: 25), GUCAUACACGGCUCUCCU (SEQ ID NO: 26), GUCAUACACGGCUCUCCUC (SEQ ID NO: 27), GUCAUACACGGCUCUCCUCU (SEQ ID NO: 28), GUCAUACACGGCUCUCCUCUC (SEQ ID NO: 30), and GUCAUACACGGCUCUCCUCUCU (SEQ ID NO: 31), wherein U can optionally be T. In some aspects, the sequence of the miRNA compound is at least 50%, at least 55%, at least 60%, at least 65%, at least 70%, at least 75%, at least 80%, at least 85%, at least 90%, or at least 95% similarity to GUCAUACACGGCUCUCCUCUCU (SEQ ID NO: 31), wherein U can optionally be T. In some aspects, the miRNA compound has a sequence that has at least 90% similarity to GUCAUACACGGCUCUCCUCUCU (SEQ ID NO: 31). In some aspects, the miRNA compound comprises the nucleotide sequence GUCAUACACGGCUCUCCUCUCU (SEQ ID NO: 31) with one substitution or two substitutions. In some aspects, the miRNA compound comprises the nucleotide sequence GUCAUACACGGCUCUCCUCUCU (SEQ ID NO: 31). Non-limiting exemplary miRNA compounds are shown elsewhere herein. In some aspects, the miRNA compound comprises at least one modified nucleotide. In some aspects, the at least one modified nucleotide is a locked nucleic acid (LNA), an unlocked nucleic acid (UNA), a bridged nucleic acid (BNA), and/or a peptide nucleic acid (PNA). In some aspects, the miRNA compound comprises a backbone modification. In some aspects, the backbone modification is a phosphorodiamidate morpholino oligomer (PMO) modification.

The methods of the present disclosure are useful for generating an animal model capable of displaying symptoms of Alzheimer's disease. Such a model can then be used in the furtherance of therapeutics to treat Alzheimer's disease or other neurodegenerative disease. In some aspects, the non-human animal model exhibits one or more symptoms of Alzheimer's disease. In some aspects, the one or more symptoms of Alzheimer's disease are selected from the group consisting of cognitive impairment and dementia. In some aspects, one or more biochemical characteristics of Alzheimer's disease are selected from the group consisting of: (i) increased amyloid beta expression in CNS, (ii) increased tau expression in CNS, (iii) increased amyloid plaques composed of amyloid-beta (Aβ) peptides, (iv) and/or neurofibrillary tangles (NFTs) composed of hyperphosphorylated tau. In some aspects, the non-human animal model shows one or more functional characteristics of Alzheimer's disease. In some aspects, the functional characteristics are selected from the group consisting of: difficulty completing tasks, repetitive behaviors, or decreased motor skills.

The methods of the present disclosure are also directed to use of a composition for preparing an Alzheimer's disease animal model comprising a miR-485-3p analog. In some aspects, the miR-485-3p may be characterized in that it is expressed in the brain and particularly the hippocampus and the cortex, though not limited to these areas. In one aspect, the miR binds to the 3′ untranslated region of SIRT1 mRNA encoding SIRT1 and inhibits its expression, thereby decreasing the SIRT1 concentration in the brain. In another aspect, the sequence of miR-485-3p may be characterized in that it is derived from a mammal, for example, a human, a mouse, a monkey, or a rat. In one aspect of the present invention, the sequence of human-derived miR-485-3p is 5′-GUCAUACACGGCUCUCCUCUCU-3′ (SEQ ID NO: 31), wherein U can optionally be T. In another aspect, the sequence is a human derived miR-485-3p comprising ACUUGGAGAGAGGCUGGCCGUGAUGAAUUCGAUUCAUCAAAGCGAGUCAUACACGGCUCUCCUCUCUUUUUUU (SEQ ID NO: 33).

In some aspects, the miR-485-3p mimic can mimic the role of miR-485-3p, thereby exhibiting an effect of increasing expression or activation of miR-485-3p. In other aspects, the miR-485-3p mimic enhances the interaction of miR-485-3p with the 3′-UTR region of SIRT1 mRNA. In some mimic, the miR-485-3p mimic can be characterized by enhancing intracellular action or function of miR-485-3p. In the present disclosure, the miR-485-3p analog can be an oligonucleotide which binds to all or a part of the nucleotide sequence UCAUACA (SEQ ID NO: 32). In some aspects, the miR485-3p mimic comprises GUCAUACACGGCUCUCCUCUCU (SEQ ID NO: 31), wherein U can optionally be T.

In some aspects, the miRNA compound can be selected from the group consisting of DNA, RNA, siRNA, shRNA, or any combination thereof. In some aspects, the miRNA compound includes DNA, RNA, polynucleotides, analogs and derivatives thereof. In some aspects, the miRNA compound is an arbitrary substance capable of mimicking the role of miR-485-3p, including a substance synthesizing the miR-485-3p sequence, small hairpin RNA molecules (shRNA), small interfering RNA molecules (siRNA), seeded target LNA (Locked Nucleic Acid) oligonucleotides, decoy oligonucleotides, an aptamer, a ribozyme, or an antibody recognizing a DNA: RNA hybrid.

In some aspects, the miRNA compound is an oligonucleotide capable of increasing the activity of miR-485-3p, including all or a part of the mature and/or mature sequence of miR-485-3p. Because miRNA binds to the target via the seed sequence, it can effectively inhibit translation of the target mRNA if the miRNA-485-3p interaction with the seed sequence is increased. In some aspects, the miRNA compound reduces the expression amount of SIRT1, increases the expression of c-Fos; induces the expression of amyloid precursor protein (APP) expression, and/or induces the production of Aβ or truncated Tau protein, and/or induces phosphorylation of Tau. In some aspects, the miRNA compound is administered to normal non-human animals and reduces the expression amount of SIRT1, increases the expression of CFOS, induces the expression of amyloid precursor protein (APP) expression, and/or induces the production of Aβ or truncated Tau protein, and/or induces phosphorylation of Tau. In some aspects, the non-human animal is a monkey, a dog, a cat, a rabbit, a guinea pig, a rat, a mouse, a cattle, or a sheep. In some aspects, the composition for preparing an Alzheimer's disease animal model is formulated and administered in the form of any one of intranasal administration, intravenous administration, subcutaneous injection, intracerebroventricular injection, inhalation administration or oral administration. In other aspects, the composition for preparing an Alzheimer's disease animal model is formulated and administered intranasally, intravenously, subcutaneously, intraperitoneally, topically, or orally.

The non-human animal model that is generated by the present methods can be tested with various methods known in the art. In some aspects, the animal models are tested for memory impairments, e.g., short-term and/or long term-memory deficits, working memory/novelty/activity, learning and memory deficits, working memory deficits, motor alterations, aggression, sleep disturbances, spatial memory deficits, and/or contextual Memory deficits. In some aspects, the animal models are tested for short-term and/or long term-memory deficits. In some aspects, the animal models are tested for working memory/novelty/activity. In some aspects, the animal models are tested for learning and memory deficits. In some aspects, the animal models are tested for working memory deficits. In some aspects, the animal models are tested for motor alterations. In some aspects, the animal models are tested for aggression. In some aspects, the animal models are tested for sleep disturbances. In some aspects, the animal models are tested for spatial memory deficits. In some aspects, the animal models are tested for contextual Memory deficits.

In some aspects, short-term and/or long term-memory deficits or working memory/novelty/activity can be tested by a Novel object recognition or object location memory. In some aspects, learning and memory deficits can be tested by water maze, Barnes maze, or hole board spatial navigation or contextual and cued fear conditioning.

In some aspects, working memory deficits can be tested by a T maze test or a Y maze test. In some aspects, motor alterations are measured by open field, gait assessment, and/or rotarod impairment. In some aspects, aggression is measured by resident-intruder male-male fighting. In some aspects, sleep disturbances are measured by spontaneous wheel running. In some aspects, spatial memory deficits are measured by radial arm maze/radial arm water maze or Barnes maze test. In some aspects, contextual Memory deficits is measured by fear conditioning test or passive avoidance task.

In some aspects, the animal models can be tested for their cognitive symptoms, e.g., attention. In some aspects, attention can be measured by a 5-Choice serial reaction time task.

Various aspects of the disclosure are described in further detail in the following subsections. The present disclosure is further illustrated by the following examples which should not be construed as further limiting. The contents of all references cited throughout this application are expressly incorporated herein by reference.

### EXAMPLES

The capability of the compositions and method of the present disclosure are discussed in the following examples. The results show that the compositions and methods of the present disclosure are capable of generating a suitable Alzheimer's disease model for the furtherance of the development of therapeutics.

### Example 1

Mirna Expression Pattern Analysis Using miRNA qPCR Array in Plasma of Alzheimer's Patients

Table 1 shows the characteristics of the patients used in the study. Four patients diagnosed with Alzheimer's disease and four control patients were evaluated. Approximately 3 ml of blood was collected in a blood tube (Becton Dickinson, Germany) supplemented with citrate (3.2% w/v) with healthy adults matched for age (within 4 years).

TABLE 1
Gender and age of normal group and patient group

| Group | Sample No | Gender | Age |
|---|---|---|---|
| Control | N1 | F | 78 |
| Control | N2 | M | 72 |
| Control | N3 | F | 74 |
| Control | N4 | M | 79 |
| Treatment | S1 | M | 72 |
| Treatment | S2 | F | 82 |
| Treatment | S3 | F | 84 |
| Treatment | S4 | M | 75 |

Samples were centrifuged at 3,500 rpm for 10 minutes to separate plasma, and stored at −80° C. until RNA extraction. MiRNA was extracted using an miRNAeasy Serum/Plasma kit (Qiagen, USA) according to the manufacturer's recommendations, and the concentration and purity of the extracted RNA were analyzed using Bioanalyzer 2100 (Agilent, USA). Eight groups were used for the study in accordance with the quality standards.

miRNA qPCR Array

The gene list used for miRNA qPCR array analysis can be found in Table 2. The maturation sequence of each miRNA can be obtained from the miRNA database (http://www.mirbase.org). The extracted RNA was screened using an miRNA arrays containing 84 different miRNAs known to be involved in the progression of Human Neurological Development and Neurological Disease.

TABLE 2
Gene List used for miRNA qPCR
Array Analysis

| No. | Mature miRNA list |
|---|---|
| 1 | hsa-let-7b-5p |
| 2 | hsa-let-7c-5p |
| 3 | hsa-let-7d-5p |
| 4 | hsa-let-7e-5p |
| 5 | hsa-let-7i-5p |
| 6 | hsa-miR-101-3p |
| 7 | hsa-miR-105-5p |
| 8 | hsa-miR-106b-5p |
| 9 | hsa-miR-107 |
| 10 | hsa-miR-124-3p |
| 11 | hsa-miR-125b-5p |
| 12 | hsa-miR-126-5p |
| 13 | hsa-miR-128-3p |
| 14 | hsa-miR-130a-3p |
| 15 | hsa-miR-132-3p |
| 16 | hsa-miR-133b |
| 17 | hsa-miR-134-5p |
| 18 | hsa-miR-135b-5p |
| 19 | hsa-miR-138-5p |
| 20 | hsa-miR-139-5p |
| 21 | hsa-miR-140-5p |
| 22 | hsa-miR-146a-5p |
| 23 | hsa-miR-146b-5p |
| 24 | hsa-miR-148b-3p |
| 25 | hsa-miR-151a-3p |
| 26 | hsa-miR-152-3p |
| 27 | hsa-miR-15a-5p |
| 28 | hsa-miR-15b-5p |
| 29 | hsa-miR-181a-5p |
| 30 | hsa-miR-181d-5p |
| 31 | hsa-miR-191-5p |
| 32 | hsa-miR-193b-3p |
| 33 | hsa-miR-195-5p |
| 34 | hsa-miR-19b-3p |
| 35 | hsa-miR-203a-3p |
| 36 | hsa-miR-20a-5p |
| 37 | hsa-miR-212-3p |
| 38 | hsa-miR-22-3p |
| 39 | hsa-miR-24-3p |
| 40 | hsa-miR-26b-5p |
| 41 | hsa-miR-27a-3p |
| 42 | hsa-miR-28-5p |
| 43 | hsa-miR-298 |
| 44 | hsa-miR-29a-3p |
| 45 | hsa-miR-29b-3p |
| 46 | hsa-miR-29c-3p |
| 47 | hsa-miR-302a-5p |
| 48 | hsa-miR-302b-5p |
| 49 | hsa-miR-30d-5p |
| 50 | hsa-miR-320a |
| 51 | hsa-miR-328-3p |
| 52 | hsa-miR-337-3p |
| 53 | hsa-miR-338-3p |
| 54 | hsa-miR-339-5p |
| 55 | hsa-miR-342-3p |
| 56 | hsa-miR-346 |
| 57 | hsa-miR-34a-5p |
| 58 | hsa-miR-376b-3p |
| 59 | hsa-miR-381-3p |
| 60 | hsa-miR-409-3p |
| 61 | hsa-miR-431-5p |
| 62 | hsa-miR-432-5p |
| 63 | hsa-miR-433-3p |
| 64 | hsa-miR-455-5p |
| 65 | hsa-miR-484 |
| 66 | hsa-miR-485-3p |
| 67 | hsa-miR-485-5p |
| 68 | hsa-miR-487a-3p |
| 69 | hsa-miR-488-3p |
| 70 | hsa-miR-489-3p |
| 71 | hsa-miR-499a-5p |
| 72 | hsa-miR-509-3p |
| 73 | hsa-miR-511-5p |
| 74 | hsa-miR-512-3p |
| 75 | hsa-miR-518b |
| 76 | hsa-miR-539-5p |
| 77 | hsa-miR-652-3p |
| 78 | hsa-miR-7-5p |
| 79 | hsa-miR-9-5p |
| 80 | hsa-miR-9-3p |
| 81 | hsa-miR-92a-3p |
| 82 | hsa-miR-93-5p |
| 83 | hsa-miR-95-3p |
| 84 | hsa-miR-98-5p |

The quantitative PCR assay method was performed. Mature miRNAs are generally 22 nucleotides in length, and are noncoding RNAs responsible for post-transcriptional regulation. Mature miRNAs were polyadenylated by poly (A) polymerase and synthesized as oligo-dT primers. The oligo-dT primers have a 3′ degenerate anchor and a universal tag sequence at the 5′ end, allowing for mature miRNA amplification during real-time PCR. A miRNA SYBR Green PCR Kit (Qiagen) was used to quantify mature miRNAs during real-time PCR.

Analysis of miRNA Expression Pattern by Volcano Plot

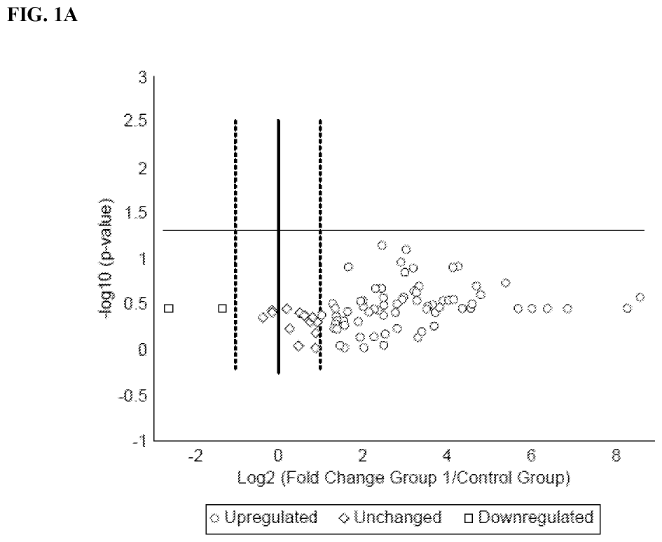

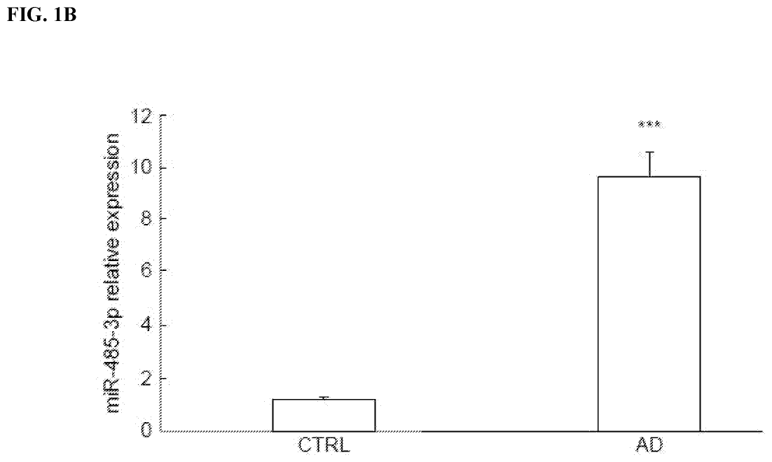

An analysis and volcano plot of miRNA expression patterns of the 84 miRNA species found in Table 2 for the patient group versus the normal group using miRNA expression pattern analysis can be seen in FIG. 1A. The x-axis represents Fold change and the y-axis represents the p-value of -log10. A horizontal black line indicates a p value of 0.05 or less. The results of the Volcano blot analysis showed that hsa-miR-105-5p, hsa-miR-98-5p, hsa-miR-15a-5p, hsa-3p, hsa-miR-92a-3p, hsa-miR-28-5p, hsa-miR-30d-5p, hsa-miR-212-3p, HSA-miR-381-3p, hsa-miR-431-5p, hsa-miR-130a-3p, hsa-miR-146b-5p, HSA-miR-223p, hsa-miR-509-3p, hsa-miR-139-5p, hsa-miR-499a-5p, hsa-miR-203a-3p, HSA-miR-128-3p, hsa-miR-487a-3p, hsa-miR-485-3p, hsa-miR-195-5p, hsa-miR-433-3p, hs-miR-191-5p, hsa-miR-489-3p, hsa-miR-432-5p, hsa-miR-29c-3p, hsa-miR-miR-106b-5p, hsa-miR-101-3p, hsa-miR-328-3p, hsa-let-7b-5p, hsa-miR-539-5p, hsa-HSA-miR-515b, hsa-miR-148b-3p, hsa-miR-181d-5p, hsa-miR-7-5p, hsa-miR-512-3p, hsa-miR-18b-5p, hsa-miR-15b-5p, hsa-miR-15b-5p, hsa-miR-34a-5p, hsa-miR-346, hsa-miR-511-5p, hsa-miR-485-3p, hsa-miR-485-5p, hsa hs-miR-489-3p, hsa-miR-499a-5p, hsa-miR-509-3p, hsa-miR-511-5p, hsa-miR-512-3p, hsa-miR HS-miR-93-5p, hsa-miR-652-3p, hsa-miR-7-5p, hsa-miR-92a-3p, and hsa-miR-98-5p expression was elevated in the patient group. However, miRNA expression changes other than hsa-miR-485-3p was not statistically significant. In the case of hsa-485-3p, the p-value is 0.00439, which is significantly increased in Alzheimer's patients compared to the normal group. When the normal group was normalized to 1, severe dementia showed a difference of about 9 fold (FIG. 1B).

### Example 2

Human miRNA Target Gene Prediction and Conservation of the Same miRNA Target Gene in Mouse

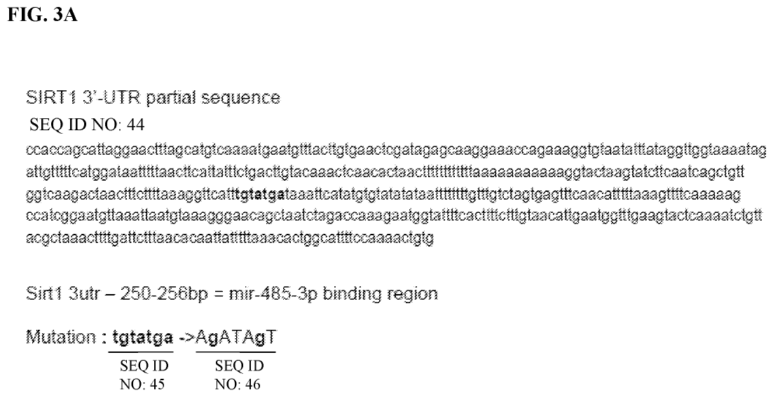

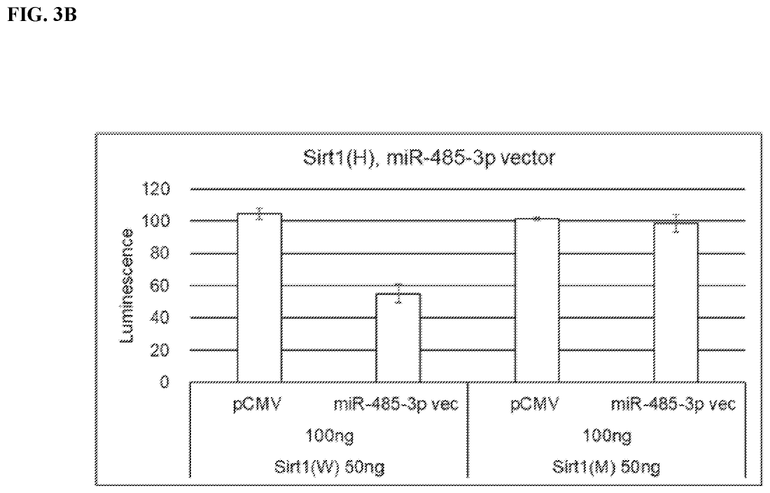

In order to analyze the base sequence and target position of hsa-miR-485-3p, the 3-terminal untranslated region (UTR) of human-derived SIRT1 was amplified using hsa-miR-485-3p and the target was confirmed (FIG. 2). It was also confirmed that the identified seed sequence was also conserved in the non-translated region of the SIRT1 3-terminal from mmu-miR-485-3p. A listing of SIRTI 3′-untranslated region (UTR) mRNA known as the target of hsa-miR485-3p, showing the target SIRT1 3′-untranslated region (UTR) mRNA of miR-485-3p can be seen in FIG. 3A. The 5′ seed sequence of miR-485-3p is found at base pairs 250-256.

TABLE 3
The nucleotide sequence of hsa-miR-485-3p

| Gene | Sequence(5′->3′) |
|---|---|
| hsa-miR-485-3p | GUCAUACACGGCUCUCCUCUCU |
| | (SEQ ID NO: 31) |

Table 3 shows the sequence of hsa-miR-485-3p, which was synthesized to investigate the physiological function of miR-485-3p using the Alzheimer's disease model.

TABLE 4
Sequence and target position
analysis of mmu-miR485-3p

| Gene | Sequence(5′->3′) |
|---|---|
| mmu-miR-485-3p | AGUCAUACACGGCUCU |
| | CCUCUC (SEQ ID NO: 34) |

| Target | | Total | Representative |
|---|---|---|---|
| gene | Gene name | sites | miRNA |
| SIRT1 | NAD-dependent | 1 | mmu-miR-485- |
| | deacetylase | | 3p |
| | sirtuin-1 | | |

Table 4 summarizes the 3′-untranslated region (UTR) and the mmu-miR-485-3p region of mouse SIRT1 using the target sequencing software (TargetScan, PicTar, and microT). It was confirmed that the target sequence of 3p was conserved. It was confirmed that the 3′-untranslated region (UTR) of mouse ELAVL2 was the target of mmu-miR-485-3p.

### Example 3

Confirmation of Binding to 3′ UTR of SIRTI among the Targets Validated in AD

The wild-type (WT) 3′-UTRs of human SIRT1 and a mutated (MT) 3′-UTR of human SIRT1 containing mutations in the predicted miR-485-3p seed regions were amplified and cloned into the psiCHECK-2 luciferase reporter vector (Promega, Madison, Wis., USA). For the luciferase activity assay, HEK293T cells were plated into 24-well plates and co-transfected with psiCHECK2-SIRT1-3′UTR-WT or psiCHECK2-SIRT1-3′UTR-MT, with pCMV-MIR (Origene) pCMV-MIR-miR-485-3p. After 48 h of transfection, firefly and Renilla luciferase activity was determined by the Dual-Luciferase Reporter Assay System (Promega). Relative Renilla luciferase activity was measured by normalizing to the firefly luciferase activity.

The results showed that the miR-485-3p seed sequence matches the 3′ UTR sequence of human SIRT1. miR-485-3p binds to the 3′ UTR sequence of SIRT1 but does not bind to the mutant 3′ UTR sequence of SIRT1 (FIG. 3B).

### Example 4

Confirmation of Cellular Expression of Target Proteins through miRNA-485-3p′s Gain of Function

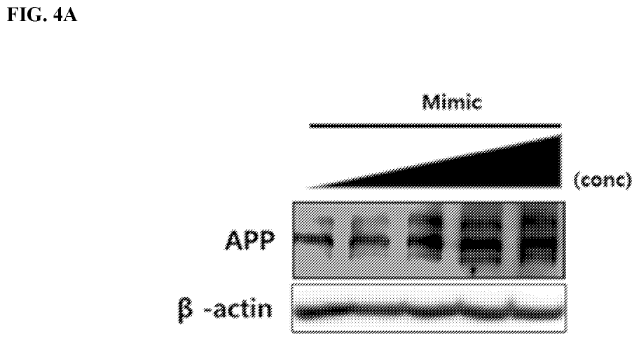

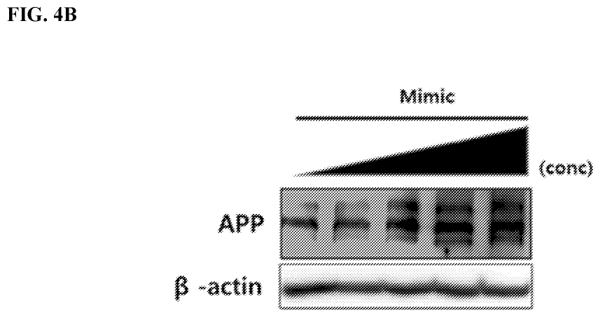

Neuro 2a, a murine neuroblastoma cell line, was transfected with 5-100 or 50 nM miR-485-3p mimic (or negative control miRNA; IDT, USA) using In Vitro Transfection Lipofectamine 2000. Cell homogenates were obtained 48 hours after transfection and western blotting was performed using an anti-APP antibody (cell signaling, USA), an anti-Tau antibody (Thermofisher SCIENTIFIC) and an anti-p-Tau antibody (Thermofisher SCIENTIFIC). Immunoreactive proteins were visualized as chemiluminescent reagents (GE health care, UK) and quantitated and quantified using a chemical image analyzer (Fusion SL). The expression of APP, Tau and p-Tau after transfection of miR-485-3p mimic in Neuro 2a cells was compared. The expression of APP was increased by the concentration of the control in the cell transfected with miR-485-3p mimic as shown in FIG. 4A. In addition, treatment of miR-485-3p mimic also increased the phosphorylation of Tau protein, another characteristic of Alzheimer's disease as shown in FIG. 4B.

Analysis of SIRT1, CFOS, APP and Aβ Proteins in Normal Mouse Brain Treated with miR-485-3p Mimic Intranasally.

Enhancement of the expression and activity of miR-485-3p was induced by nasal administration of sequence-specific analogs. The intranasal administration of the analogs was carried out according to a method of targeting the brain without anesthetizing the mice (Leah RT, et al. (2013) Intranasal Administration of CNS Therapeutics to Awake Mice. J Vis Exp. 4440). After completing the adaptation step described in the above paper, an intranasal grip is applied to the mouse and the abdomen is directed upward, and the pipette is placed in front of one nasal cavity. 6 μl is inhaled into the form of a drop with a pipette (1 drop=3 μl). After holding the mouse steady for a 15 second period, the procedure is then performed on the other nasal cavity. After 2 minutes, the same procedure is repeated for a total inhaled volume of 24 μl (miR-485-3p mimic-5′-GUCAUACACGGCUCUCCUCUCU-3 (SEQ ID NO: 35); 24 μl of 0.1% v/v diethyl pyrocarbonate-5 nmol in distilled water treated; Bioni, Korea).

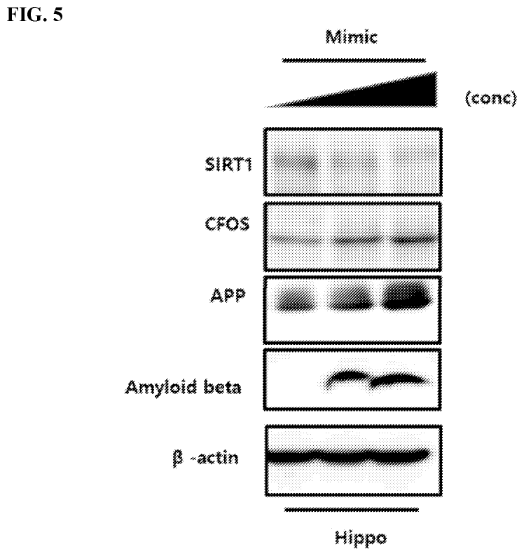

Control mice were dosed with equal volume of Vehicle. Four weeks after nasal administration, the anesthetized mice were sacrificed and the brains were immediately removed. A sample of the brain (hippocampus and cortex) was prepared and immunized with ELAVL2 antibody (abcam, USA), Aβ42 antibody (cell signaling, USA), APP antibody (cell signaling, USA), Tau antibody (Thermofisher SCIENTIFIC), and p-Tau antibody (Thermofisher SCIENTIFIC). Immunoreactive proteins were visualized as chemiluminescent reagents (GE health care, UK) and quantitated and quantified using a chemical image analyzer (Fusion SL). FIG. 5 shows the results of analysis of SIRT1, CFOS, APP and Aβ in the brain of normal mice treated with miR-485-3p mimic intranasally. In normal mice treated with miR-485-3p mimic, SIRT1 was decreased in a concentration-dependent manner, and APP and Aβ 42 were increased (FIG. 5).

### Example 5

Analysis of Amyloid Beta (Aβ) 42 and Amyloid Beta (Aβ) Oligomers in Normal Mouse and Dementia Animals (5xFAD) Intranasally Treated with miR-485-3p Mimic

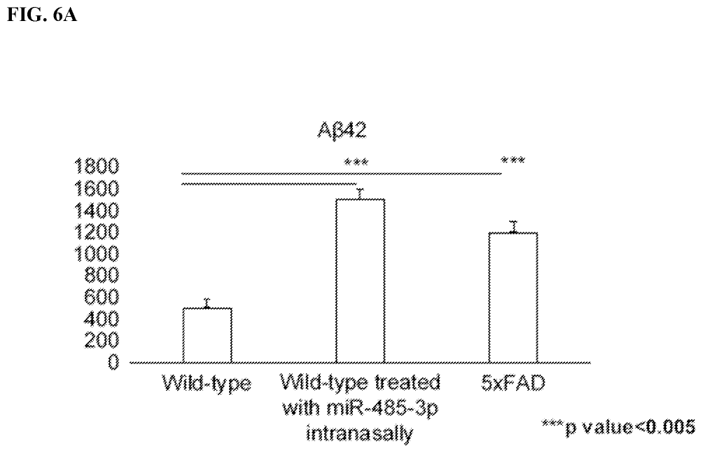

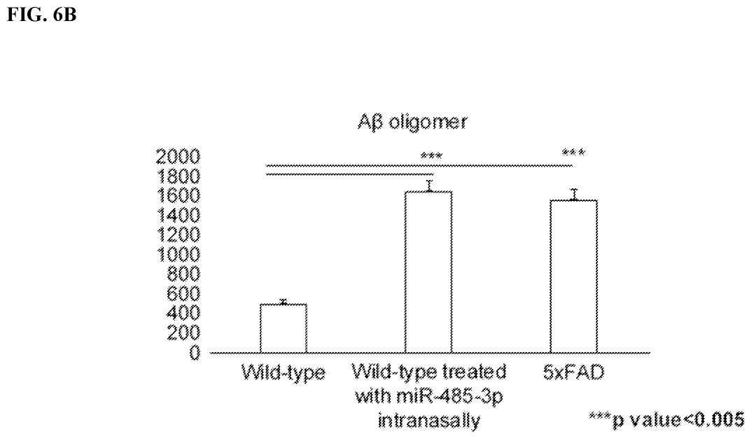

Aβ 42 and Aβ oligomers were measured using a mouse amyloid beta (1-42) assay kit (IBL) and the manufacturer's instructions. Aβ 42 and Aβ oligomers were analyzed in the hippocampus of normal mice and demented animals (5×FAD), which were intranasally treated with miR-485-3p mimic in the same manner as in Example 4 above. It was confirmed that the treatment of miR-485-3p mimic affects the production of Aβ 42 and the production of Aβ oligomers (FIG. 6A and FIG. 6B). Therefore, we confirmed the viability of a model of Alzheimer's dementia in normal mice treated with miR-485-3p mimic.

### Example 6

Analysis of Species miRNA Sequence Homology

Sequence similarity of miRNA485-3p among different species was examined by performing multiple sequence alignment using BIOEDIT. As shown in FIG. 7, sequence similarity between species was confirmed, suggesting conservation of miRNA-485-3p's functionality.

### Example 7

Comparison of Cognitive Function of the Inducible Mouse with miR-485-3p Mimic and Genetically Modified Mouse (5xFAD)

An animal model of Alzheimer disease with intraneuronal accumulation of Aβ 42 Y-maze experiment was conducted. A miR-485 mimic (miR-485-3p mimic-5′-GUCAUACACGGCUCUCCUCUCU-3 (SEQ ID NO: 35)) was administered to mice (n=5) at a dose of about 2 mg/kg, at a dose of about 4 mg/kg, at a dose of about 10 mg/kg, at a dose of about 20 mg/kg after being diluted in 200 μl buffer. The miR-485 mimic was administered once a week for 1 month by intravenous injection. The miR-485 mimic (miR-485-3p mimic-5′-GUCAUACACGGCUCUCCUCUCU-3 (SEQ ID NO: 35)) was also administered intrathecally to mice (n=5) at a dose of 0.075 mg/kg, 0.15 mg/kg, 0.35 mg/kg, or 0.75 mg/kg using a 10 μl Hamilton syringe (26-gauge blunt needle) once a week for 1 month. The miR-485 mimic (miR-485-3p mimic-5′-GUCAUACACGGCUCUCCUCUCU-3 (SEQ ID NO: 35)) was also administered intracerebroventricularly (ICV) to mice (n=5) at a dose of 0.075 mg/kg, 0.15 mg/kg, 0.35 mg/kg, or 0.75 mg/kg once a week for 1 month. Intracerebroventricular (ICV) position was identified using the coordinates from the bregma: AP=−0.2 mm, L=±1.0 mm, ventral (V)=−2.5 mm.

Overexpression of normal and mutant forms of APP and PSEN1 in the intranasally treated miR-485-3p mimic led to overcorrection of the cognitive function of the genetically engineered mouse (5xFAD).

The Y-maze experimental system consists of a Y-shaped, labyrinth made of black acrylic plates (10 cm by 41 cm, and 25 cm by 25 cm), and each labyrinth is arranged at an angle of 120°. After setting each labyrinth as A, B, and C arms, the animals are carefully placed in one arm to allow them to move freely for 8 minutes. Measurements are taken based on the number and order of entry into each labyrinth arm in order to determine the spontaneous alteration (%). Each time the test subject enters a new arm, an entry is recorded. An alteration occurs when a test subject visits all three areas sequentially (i.e., ABC, BCA, CAB, etc.) The % spontaneous alteration was calculated using the following equation:

% Spontaneous alteration=total number of alterations/(total number of entries−2)×100

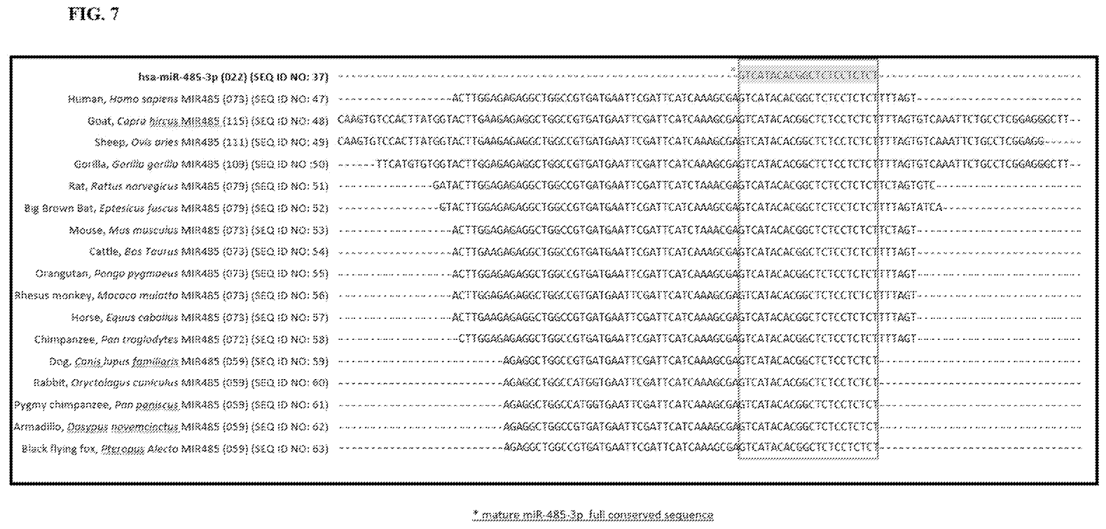

FIG. 8 shows the results of cognitive function comparisons of normal mice and mice with symptoms of dementia (5xFAD) and wild-type animals treated with miR-485-3p mimic. In the normal mice and 5xFAD-control, the altering activity decreased as subjects with reduced neural function will be less likely to visit arms that they have not recently visited. Because the main symptoms of Alzheimer's dementia are behavioral and memory impairment, behavioral disorders of substance-treated normal mice and 5xFAD appear to be due to excessive accumulation and pathology of Aβ. This result shows that administration of miR-485-3p mimic promotes the production of Aβ 42, which leads to pathological symptoms such as behavioral disturbances and memory depression caused by Alzheimer's disease, and may lead to the other major symptoms of Alzheimer's disease. Therefore, animals with pathological, symptomatic characteristics induced by modulation of miR-485-3p within a short period of time show potential as a new experimental animal for a group studying Alzheimer's dementia, particularly Aβ and p-Tau.

A t-test was used to compare the two groups and to compare three or more groups using the Krushall-Wallis analysis of variance. When the P-value obtained from the Krushall-Wallis test was less than 0.05, post-hoc testing of the intergroup comparisons was performed using the Mann-Whitney U test. Two-tailed P-values of less than 0.05 were considered statistically significant.

### Example 9

miR-485-3p Lentiviral Vector Construction, Production, and Titration

Lentivirus expressing GFP was purchased from Applied Biological Materials Inc. (abm) (pLenti-III-mir-GFP Cloning Vector. CAT.NO m016, Canada. Lentivirus expressing miR-485-3p was constructed with the miR485-3p mature sequence (GTCATACACGGCTCTCCTCTCT) (SEQ ID NO: 37).

293T cells were plated on 10 cm culture plates at 3×10⁶ and cultured until confluency of 70-80% was achieved. 2 hours before transfection of viral DNA, fresh DMEM was added to the cells. The cells were then mixed with the following DNA—transfer vector 10 μg+pMDL g/pRR 5 μg+pRSV-Rev 2.5 μg+pMD2.G 2.5 μg+Opti-MEM 1 mL based on one 10 cm plate—and were incubated for 20 minutes. The cells were treated with 35 μl of TransIT-lenti and DNA mixture and cultured for 72 hours. The culture media containing virus were harvested from the cells. Cell debris was spun down by centrifuge at 6,000 rpm for 15 minutes. The supernatant was collected and centrifuged at 29,000 rpm and ultra-centrifuged for 2 hours to obtain virus pellets. The pellets were diluted in cold PBS with 1/1000 volume of virus supernatant, aliquoted, and stored in −80° C. deep freezer. The virus titer was measured using a lenti-X p24 titer kit.

### Example 10

Lentiviral Expression of miR-485-3p in Mouse Hippocampus and Analysis of Cognitive Function

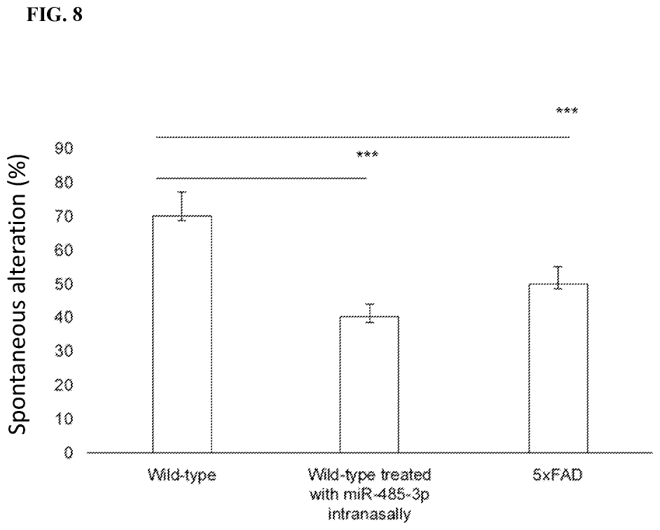

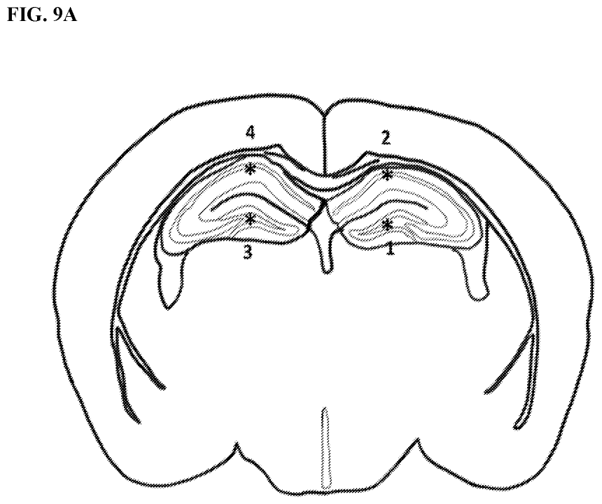

Six-week-old mice were anesthetized with intraperitoneal injection using an anesthetic (Tribromo ethyl alcohol+2-Methyl-2-butanol+d.w). The dentate gyrus and CA1 region (AP: −2 mm/ML: ±1.5 mm/DV: −2.7 & −2.0) in the mice's hippocampus were injected with lenti-virus using a stereotaxic surgery equipment and Hamilton syringe 700 series. The virus was injected in two locations per hemisphere (FIG. 9A). The virus volume per site was 1.5 μl, and the injection flow rate was 0.2 μl/min. The injection needle was left in the site for 15 minutes to make sure that the injected viruses were sufficiently delivered. After the surgery, the body weight of the mice was maintained to determine any health problems caused by the surgery. After the mice were raised to express the injected virus for one month, the mice were placed under the Novel Object Recognition Tests (NORT).

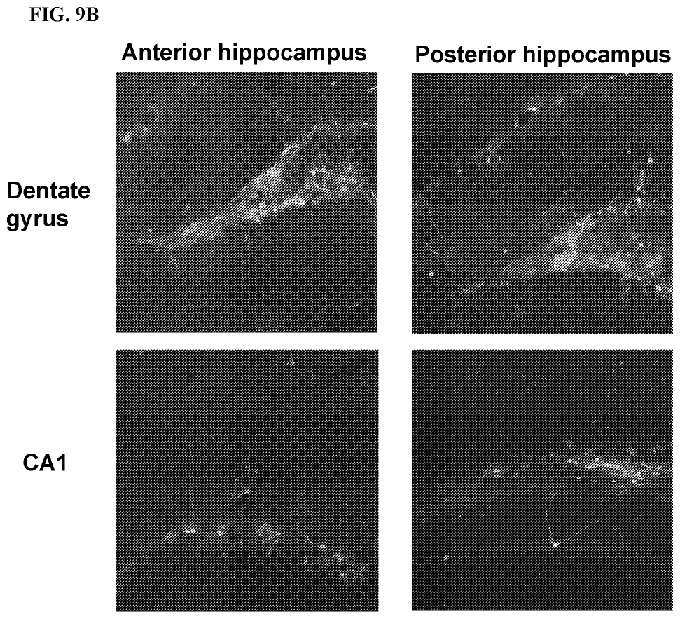

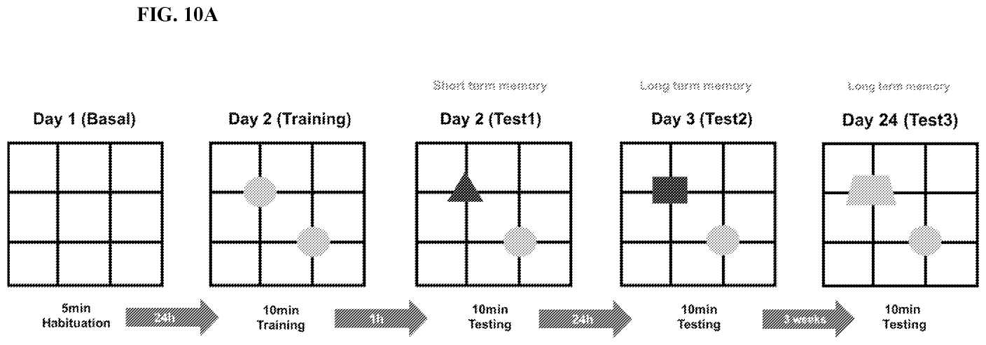

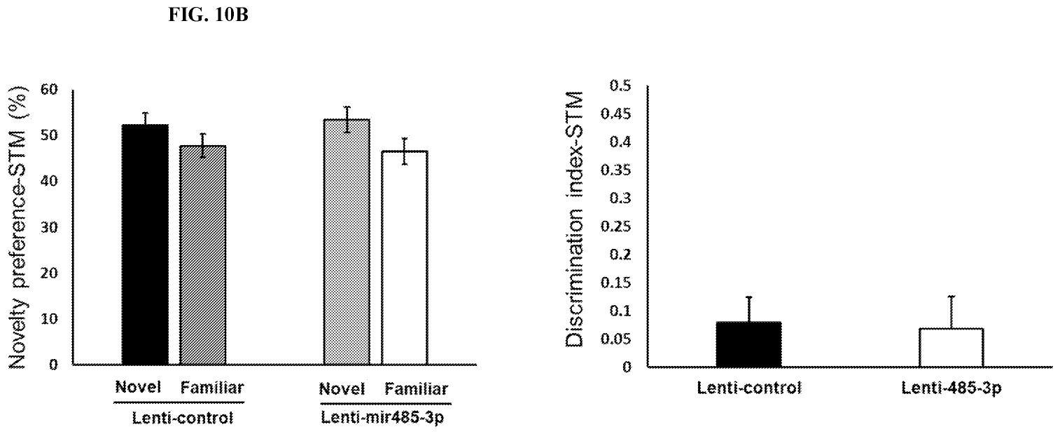

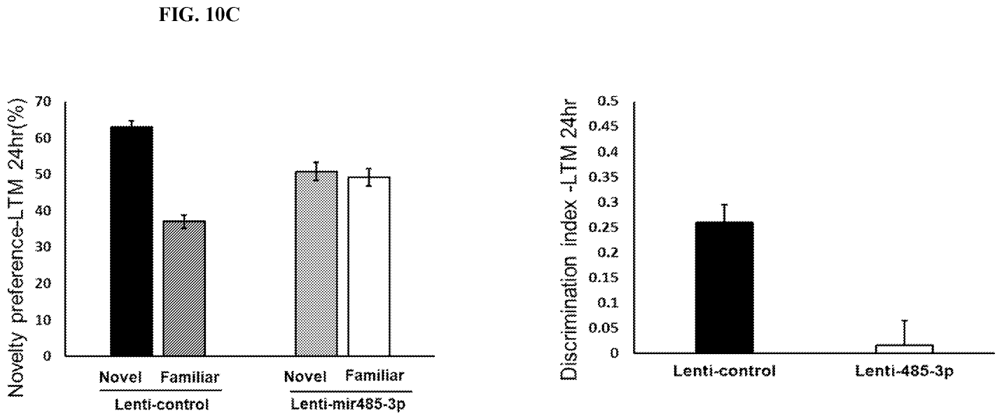

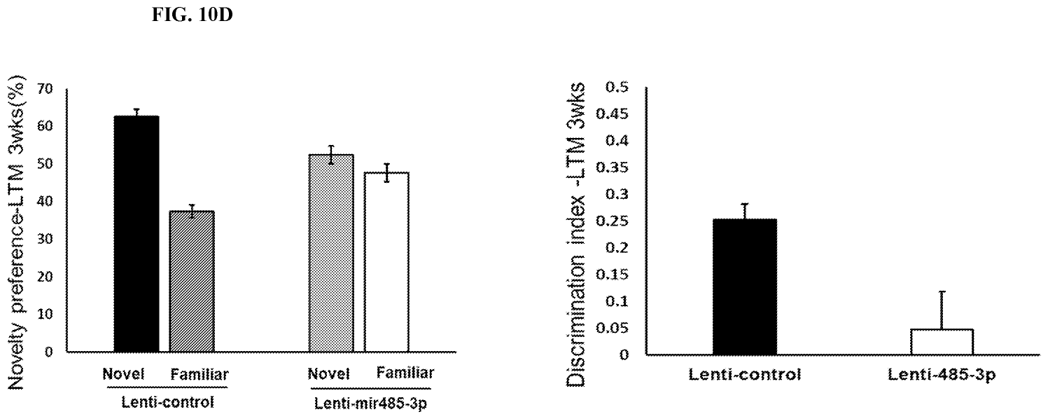

NORT was performed in a white matte chamber with dimensions of 450×450×450 mm. The mice (lenti-miR-485-3p and lenti-control treated) were allowed to move freely for 5 minutes on day 1, allowing them to adapt to the space. After 24 hours, the same two objects (e.g., A&A) were placed in the first and fourth quarters of the chamber and the mice were allowed to move freely for 10 minutes to learn the two objects and the space. After one hour, to measure short term memory, one of the two objects was changed to a different shape and color (e.g., A&B). The curiosity of the new object was measured by number of nose pokes. To measure long term memory, after 24 hours and 3 weeks, one of the two objects was changed to a different shape and color in the same way (e.g., 24 hours—A&C/3 weeks—A&D), and the number of nose pokes were measured (FIG. 10A). The results were analyzed by comparing the number of nose pokes to new and familiar objects per group, and discrimination index was calculated as follows: [(novel-familiar)/(novel+familiar)] (FIG. 10B-D).

Compared to the control group, there was no difference in short-term memory after 1 hour of training in the group overexpressed with miR485-3p (FIG. 10B), but a statistically significant difference was observed in long term memory tested after 24 hours (FIG. 10C) and 3 weeks later (FIG. 10D). In the case of the control group, it was observed that the new object was recognized and the mice were curious, whereas the miR-485-3p overexpression group did not recognize the new object and no difference in response to the familiar object was observed. This suggests that miR-485-3p overexpression in hippocampus weakens long term memory formation.

After the NORT was completed, the mice were sacrificed through cardiac perfusion. The brains of the mice were removed and fixed at 4° C. for 4 hours in 4% paraformaldehyde. Subsequently, 30% sucrose in 0.1M PBS was added to the brains for about 48 hours, and embedding (O.C.T compound) was performed to cryosection to a thickness of 40 μm. Nuclei were stained with DAPI (1:500), mounted with a hardset antifade medium, and photographed using confocal microscopy. miR-485-3p was expressed in both the anterior and posterior of the hippocampus (FIG. 9B).

All mice used in the experiments were wild type male c57BL6/J mouse lines, and 6-week-old mice purchased through the Bio link to Korea. Mice undergoing surgical and behavioral experiments were bred in a single cage to exclude physical injuries or psychological anxiety caused by other male attacks, and water and feed were provided as ad libitum, in a 12 hour light/dark cycle environment. Mice injected with Lenti-control virus and Lenti-mir485-3p virus into the brain were checked for health status after surgery, with regular bodyweight checks and confirmation that there was no physical abnormality. All behavioral experiments were conducted in the light phase, and all mice in each group were tested under the same conditions.

GraphPad Prism 8 was used for all statistical analysis, and statistical significance between two groups was analyzed by two-way ANOVA and unpaired t-test.

1. A method of preparing a non-human animal model for Alzheimer's disease comprising administering to a non-human animal a compound that mimics miR-485 (miRNA compound).

2. The method of claim 1, wherein the miRNA compound comprises a nucleotide sequence comprising 5′ UCAUACA 3′ (SEQ ID NO: 32) and wherein the miRNA compound comprises about 6 to about 30 nucleotides in length.

3-5. (canceled)

6. The method of claim 1, wherein the miRNA compound comprises a nucleotide sequence selected from the group consisting of: GUCAUACA (SEQ ID NO: 1), UCAUACAC (SEQ ID NO: 2), UCAUACACG (SEQ ID NO: 3), UCAUACACGG (SEQ ID NO: 4), UCAUACACGGC (SEQ ID NO: 5), UCAUACACGGCU (SEQ ID NO: 6), UCAUACACGGCUC (SEQ ID NO: 7), UCAUACACGGCUCU (SEQ ID NO: 8), UCAUACACGGCUCUC (SEQ ID NO: 9), UCAUACACGGCUCUCC (SEQ ID NO: 10), UCAUACACGGCUCUCCU (SEQ ID NO: 11), UCAUACACGGCUCUCCUC (SEQ ID NO: 12), UCAUACACGGCUCUCCUCU (SEQ ID NO: 13), UCAUACACGGCUCUCCUCU (SEQ ID NO: 14), UCAUACACGGCUCUCCUCUC (SEQ ID NO: 15), UCAUACACGGCUCUCCUCUCU (SEQ ID NO: 16), GUCAUACAC (SEQ ID NO: 17), GUCAUACACG (SEQ ID NO: 18), GUCAUACACGG (SEQ ID NO: 19), GUCAUACACGGC (SEQ ID NO: 20), GUCAUACACGGCU (SEQ ID NO: 21), GUCAUACACGGCUC (SEQ ID NO: 22), GUCAUACACGGCUCU (SEQ ID NO: 23), GUCAUACACGGCUCUC (SEQ ID NO: 24), GUCAUACACGGCUCUCC (SEQ ID NO: 25), GUCAUACACGGCUCUCCU (SEQ ID NO: 26), GUCAUACACGGCUCUCCUC (SEQ ID NO: 27), GUCAUACACGGCUCUCCUCU (SEQ ID NO: 28), GUCAUACACGGCUCUCCUCUC (SEQ ID NO: 30), and GUCAUACACGGCUCUCCUCUCU (SEQ ID NO: 31).

7. The method of claim 1, wherein the miRNA compound comprises a nucleotide sequence that has at least about 50% sequence identity to GUCAUACACGGCUCUCCUCUCU (SEQ ID NO: 31).

8-10. (canceled)

11. The method of claim 1, wherein the miRNA compound comprises (i) at least one modified nucleotide, (ii) a backbone modification, or (iii) both (i) and (ii).

12-14. (canceled)

15. The method of claim 1, wherein the miRNA compound is administered to the non-human animal intrathecally, intracerebroventricularly, or parenterally.

16. (canceled)

17. The method of claim 15, wherein the miRNA compound is administered at a dose of about 0.01 mg/kg to about 20 mg/kg.

18-20. (canceled)

21. The method of claim 1, wherein the miRNA compound is administered to the non-human animal about every 12 hours to about every five weeks.

22. The method of claim 1, wherein the miRNA compound is administered to the non-human animal for a duration of about 1 month to about 1 year.

23. The method of claim 1, wherein the miRNA compound is administered in a delivery agent, a viral vector, or both.

24-30. (canceled)

31. The method of claim 1, wherein, after the administration, the non-human animal model exhibits: (i) one or more symptoms of Alzheimer's disease, (ii) one or more biochemical characteristics of Alzheimer's disease, (iii) one or more functional characteristics of Alzheimer's disease, or (iv) any combination of (i) to (iii).

32-36. (canceled)

37. The method of claim 1, further comprising screening an agent for its therapeutic effect in treating Alzheimer's disease in the non-human animal model.

38-42. (canceled)

43. The method of claim 37, further comprising administering a therapeutically effective amount of the agent which has been screened in the non-human animal to a human subject in need thereof.

44. (canceled)

45. A non-human animal model prepared by the method of claim 1.

46. A non-human animal model for Alzheimer's disease comprising a compound that mimics miR-485 (miRNA compound).

47-49. (canceled)

50. The non-human animal model of claim 46, wherein the miRNA compound comprises a nucleotide sequence comprising 5′ UCAUACA 3′ wherein the nucleotide sequence comprises about 6 to about 30 nucleotides.

51-52. (canceled)

53. The non-human animal model of claim 46, wherein the miRNA compound has a sequence selected from the group consisting of: GUCAUACA (SEQ ID NO: 1), UCAUACAC (SEQ ID NO: 2), UCAUACACG (SEQ ID NO: 3), UCAUACACGG (SEQ ID NO: 4), UCAUACACGGC (SEQ ID NO: 5), UCAUACACGGCU (SEQ ID NO: 6), UCAUACACGGCUC (SEQ ID NO: 7), UCAUACACGGCUCU (SEQ ID NO: 8), UCAUACACGGCUCUC (SEQ ID NO: 9), UCAUACACGGCUCUCC (SEQ ID NO: 10), UCAUACACGGCUCUCCU (SEQ ID NO: 11), UCAUACACGGCUCUCCUC (SEQ ID NO: 12), UCAUACACGGCUCUCCUCU (SEQ ID NO: 13), UCAUACACGGCUCUCCUCU (SEQ ID NO: 14), UCAUACACGGCUCUCCUCUC (SEQ ID NO: 15), UCAUACACGGCUCUCCUCUCU (SEQ ID NO: 16), GUCAUACAC (SEQ ID NO: 17), GUCAUACACG (SEQ ID NO: 18), GUCAUACACGG (SEQ ID NO: 19), GUCAUACACGGC (SEQ ID NO: 20), GUCAUACACGGCU (SEQ ID NO: 21), GUCAUACACGGCUC (SEQ ID NO: 22), GUCAUACACGGCUCU (SEQ ID NO: 23), GUCAUACACGGCUCUC (SEQ ID NO: 24), GUCAUACACGGCUCUCC (SEQ ID NO: 25), GUCAUACACGGCUCUCCU (SEQ ID NO: 26), GUCAUACACGGCUCUCCUC (SEQ ID NO: 27), GUCAUACACGGCUCUCCUCU (SEQ ID NO: 28), GUCAUACACGGCUCUCCUCUC (SEQ ID NO: 30), and GUCAUACACGGCUCUCCUCUCU (SEQ ID NO: 31).

54-74. (canceled)

75. A method of screening an agent for treatment of Alzheimer's disease comprising administering the agent to the non-human animal model of claim 46.

76. The method of claim 75, which further comprises monitoring the non-human animal model after the administration.

77. (canceled)

78. The method of claim 1, wherein the non-human animal comprises a goat, sheep, gorilla, rat, big brown bat, mouse, cattle, Orangutan, Rhesus monkey, horse, chimpanzee, dog, rabbit, Pygmy chimpanzee, nine-banded armadillo, or black flying fox.

79. (canceled)
